(19) United States
(12) Patent Application Publication
(10) Pub. No.: US 2025/0271423 A1
(43) Pub. Date: Aug. 28, 2025

(54) POLYMER CONJUGATE FOR BLOCKING OF NON-SPECIFIC INTERACTIONS IN IMMUNOCHEMICAL ASSAYS, METHOD OF ITS SYNTHESIS AND USE THEREOF

(71) Applicants: USTAV MAKROMOLEKULARNI CHEMIE AV CR, V.V.I., Praha 6 (CZ); I&I PRAGUE S.R.O., Praha 6 (CZ); ELISA DEVELOPMENT S.R.O., Litomerice (CZ)

(72) Inventors: Tomas ETRYCH, Klinec (CZ); Vladimir SUBR, Melnik (CZ); Libor KOSTKA, Revnicov (CZ); Jiri MOOS, Praha 3 (CZ); Jan PLICKA, Litomerice (CZ)

(21) Appl. No.: 18/858,596

(22) PCT Filed: Apr. 22, 2023
(86) PCT No.: PCT/CZ2023/050020

§ 371 Date: Oct. 21, 2024

(30) Foreign Application Priority Data
Apr. 22, 2022 (CZ) PV 2022-167

(51) Int. Cl. G01N 33/543; C08F 220/18; C08F 220/56; C08F 220/60

(52) U.S. Cl. CPC G01N 33/54393; C08F 220/1803; C08F 220/56; C08F 220/603

(57) ABSTRACT

The present invention relates to macromolecular blockers of non-specific interactions of an analyte and other components of the analytical system with the solid phase, which considerably decreases the non-specific interactions during the assay. The macromolecular blocker comprises synthetic copolymer or homopolymer, selected from the group comprising polyacrylamide, polymethacrylamide, polyacrylate, polymethacrylate, poly(N-(2-hydroxypropyl)methacrylamide), poly(2-oxazoline); to which at least one hydrophobically-active anchor is attached via a covalent bond, said hydrophobically-active anchor enabling suppression of non-specific interactions in immuno assays. The invention further relates to a method of preparing and to the use of said macromolecular blockers for capturing of specific antibodies or antigens or other molecules on the surface of the solid phase. The present invention enables to replace animal-derived proteins with a synthetic macromolecular blocker that is not only more effective in blocking activity itself, but it is also defined in its structure, has high batch-to-batch reproducibility, and does not need to be tested for the presence of viruses and other pathogens.

### FIELD OF ART

The present invention describes synthetic macromolecular blockers of non-specific interactions during immunochemical assays and adherent anchors that allow immunoassays to be performed without the presence of commonly used blocking proteins.

### BACKGROUND ART

One of the key problems of any immunochemical determination, i.e. a determination using the interaction of an analyte with a specific antibody, immunoglobulin, is the problem of non-specific binding of individual reactants (analyte, other components from the sample, antibody and analyte-antibody complex) to the surface of the solid phase used as part of the system or to the surface of the reaction vessel. The problem of non-specific binding (NSB) leads to the deterioration of some determination parameters (sensitivity, specificity and reproducibility). To minimise NSB, inert, i.e. indifferent to the immunochemical reaction itself, reactants that bind to the solid phase non-specifically and thus minimise the analogous non-specific binding of molecules participating in the immunochemical reaction or detection are most often used.

Historically, the most commonly used NSB blockers are proteins of animal origin, namely bovine serum albumin (BSA), casein, skim milk, fish or pig gelatine, etc. A common disadvantage of these animal-derived blockers is the considerable variability of individual production batches, requiring costly testing of each new batch by end-users who use these blockers for their standardised production. A further disadvantage associated with using input material of animal origin is the requirement for tests to demonstrate safety regarding the content of animal pathogens or contaminants listed by the relevant legislation.

Despite the above caveats, BSA can be identified as the most used blocker of non-specific interactions. For immunochemical tests, this is the so-called ‘Albumin Fraction V’, isolated in a way that destroys the enzymatic activities of proteases. BSA is not only used as an NSB blocker, but quite a few diagnostic systems use BSA conjugated with biotin to bind avidin or streptavidin and subsequently to the binding of other biotinylated components such as antibodies or antigens.

The use of avidin or streptavidin as a ‘cross’ reactant due to its four biotin binding sites is advantageous for the construction of a relatively robust solid phase. However, BSA is certainly not an ideal molecule optimised for suppressing these non-specific interactions, either by its sorption properties or by the properties that result from the fact that BSA is of animal origin.

Synthetic molecules such as detergents like Tween-20 or polymers like polyvinyl alcohol or Ficoll have been used as alternatives to protein blocking agents (Rodda, D. J. and H. Yamazaki, Poly(vinyl alcohol) as a blocking agent in enzyme immunoassays. Immunological Investigations, 1994. 23(6-7): p. 421-428; Gardas, A. and A. Lewartowska, Coating of proteins to polystyrene ELISA plates in the presence of detergents. Journal of Immunological Methods, 1988. 106(2): p. 251-255; Steinitz, M., Quantitation of the blocking effect of Tween 20 and bovine serum albumin in ELISA microwells. Analytical Biochemistry, 2000. 282(2): p. 232-238; Lim, C. S., et al., On optimizing the blocking step of indirect enzyme-linked immunosorbent assay for Epstein-Barr virus serology. Clinica Chimica Acta, 2013. 415: p. 158-161), but often do not prevent non-specific binding to a sufficient extent (Huber, D., et al., Effectiveness of natural and synthetic blocking reagents and their application for detecting food allergens in enzyme-linked immunosorbent assays. Analytical and Bioanalytical Chemistry, 2009. 394(2): p. 539-548). Therefore, there is a demand for better blocking techniques.

Blocking agents have been described that are structurally based on cationic surfactants based on structurally different poly(ethylene glycols) conjugated with alkylamines (Fujimoto, N., et al., Polyethylene glycol-conjugated alkylamines—A novel class of surfactants for the saturation of immunoassay solid phase surfaces. Talanta, 2020. 211; Fujimoto, N., et al., Novel synthetic blocking reagents. 2010: EP 2261662 A1). In addition to modified PEGs, the use of polyvinyl alcohol has been reported to reduce the amount of non-specific antibody binding and polyvinylpyrrolidone increases the sensitivity of antibody detection (Studentsov, Y. Y., et al., Enhanced enzyme-linked immunosorbent assay for detection of antibodies to virus-like particles of human papillomavirus. Journal of Clinical Microbiology, 2002. 40(5): p. 1755-1760).

### SUMMARY OF THE INVENTION

The present invention relates to synthetic macromolecular blockers of non-specific interactions in immunoassays and similar diagnostic tests capable of suppressing non-specific sorption of the antibodies or other macromolecules used onto a solid phase. The invention describes molecules based on polyHPMAs, poly(2-oxazolines), polyacrylamides, polymethacrylamides, polyacrylates or polymethacrylates that may be useful to replace BSA and to suppress non-specific interactions in diagnostic assays. The invention further describes the use of synthetic macromolecules as highly efficient adherence anchors replacing the blocking proteins used in the adhesion component of diagnostic tests. Polymer conjugates according to the present invention are highly biocompatible, non-immunogenic, non-toxic, and highly soluble in aqueous solutions and allow attachment of molecules of only one type as well as combinations of different types with different functions. Controlled radical copolymerisation also enables the preparation of telechelic polymers bearing two different functional groups at the ends of their chains.

The present invention describes a water-soluble synthetic polymer conjugate (macromolecular blocker of non-specific interactions) comprising functional components, in particular a ‘hydrophobically active’ anchor and a ligand that reduces non-specific non-covalent interactions in biological media (‘interaction-reducing ligand’), which serve as synergistic components of the system and enhance the blocking activity of the macromolecular blocker of non-specific interactions. The primary function of the described blocker is its high activity in effectively blocking the solid phase surface and in blocking non-specific interactions in solution, thereby significantly reducing the complex non-specific interaction of the analyte with the solid phase surface. This reduction in non-specific sorption decreases the noise signal while increasing the analyte signal-to-noise ratio, significantly improving analytical precision. Preferably, the described macromolecular blocker can anchor a bio-specific molecule, e.g., avidin or streptavidin, and then bind another component reacting with the bio-specific component, e.g., biotinylated, such as antibodies or antigens. In this case, the macromolecular blocker further comprises a bio-specific anchor, preferably biotin. Preferably, two variants of the macromolecular blocker can be combined; a blocker not containing the bio-specific anchor and a blocker containing the bio-specific anchor. The latter blocker allows the binding of the bioactive molecule, e.g. avidin, to the solid phase. The first blocker, which does not contain the bio-specific anchor, is responsible for the perfect shielding of non-specific interactions of the analyte with the surface of the solid phase. The backbone of the macromolecular blocker comprises a synthetic copolymer to which functional components are covalently attached: (i) at least one ‘hydrophobically active” anchor to allow reduction of non-specific interactions in immunoassays; (ii) preferably an ‘interaction-reducing ligand’ to increase the blocking activity of the entire blocker; (iii) in some cases, the copolymer may preferably further comprise a bio-specific anchor.

The object of the present invention is a polymer conjugate functioning as a macromolecular blocker of non-specific interactions in biological media, which comprises a basic linear polymer (homopolymer or copolymer), wherein the basic linear polymer is selected from the group comprising poly[N-(2-hydroxypropyl)methacrylamide], poly(2-oxazoline), poly(acrylamide), poly(methacrylamide), poly(acrylate), poly(methacrylate), and statistical copolymers thereof. Preferably, the basic linear polymer is poly[N-(2-hydroxypropyl)methacrylamide] or poly(2-oxazoline). The polymer conjugate further comprises at least one hydrophobically active anchor of the general formula —X′—R², which is attached as a side chain via the X′ group to the carbonyl group of the monomer unit of the basic linear polymer and/or is attached via the X′ group to at least one end of the basic linear polymer.
Substituent X′ is selected from the group consisting of —NH—(CH₂)ₙ—C(═O)—; —(CH₂)ₚ—C(═O)—; covalent bond; —S—C(═S)—;
NH—(CH₂—CH₂—O)ₒ—CH₂—CH₂—C(═O)—; —O—(CH₂—CH₂—O)ₒ—CH₂—CH₂—C(═O)—; or
—NH—(CH₂)_{q}(C(═O)—NH—(CH₂)ᵣ)ₚC(═O)—; wherein n is an integer from 1 to 7; o is an integer from 1 to 15; p, q and r are independently selected from the group consisting of 1, 2 and 3;
   wherein the —CH₂— groups of X′ may be further independently substituted with one or more of the same or different side chains of a natural amino acid;
R² is selected from the group consisting of —NH—(CH₂)_{b}—CH₃; —NH—(CH₂)_{b}—(CH═CH—CH₂)ₐ—(CH₂)_{b}—CH₃; —S—(CH₂)_{b}—(CH═CH—CH₂)ₐ—(CH₂)_{b}—CH₃; —O—C(═O)—(CH₂)_{b}—CH₃; —NH—C(═O)—(CH₂)_{b}—CH₃; phenyl, —S—(CH₂)ₐ—CH—((CH₂)_{b}—CH₃)₂; —O—(CH₂)_{b}—CH₃; —O—(CH₂)ₐ—C—((CH₂)_{b}—CH₃)₂; —NH—(CH₂)ₐ—C—((CH₂)_{b}—CH₃)₂; —O—(CH₂)_{b}—(CH═CH—CH₂)ₐ—(CH₂)_{b}—CH₃; and —O—C(═O)—(CH₂)ₐ—CH—((CH₂)_{b}—CH₃)₂; wherein a is an integer from 0 to 4, b is an integer from 4 to 17;
wherein, if X′ is not a covalent bond, then R² group is bound through its end —S— or —NH— or —O— group or via carbon (in the case of phenyl) to the end carbonyl or thiocarbonyl group of the X′ linker;
   wherein the hydrophobically active anchor of the general formula —X′—R² is attached via the X′ group as a side chain to the carbonyl group of the monomer unit of the basic linear polymer;
   and/or
   the hydrophobically active anchor of the general formula —X′—R² is attached via the X′ group to at least one end of the basic linear polymer;
   and wherein the end groups of the basic linear polymer are independently selected from the group consisting of:

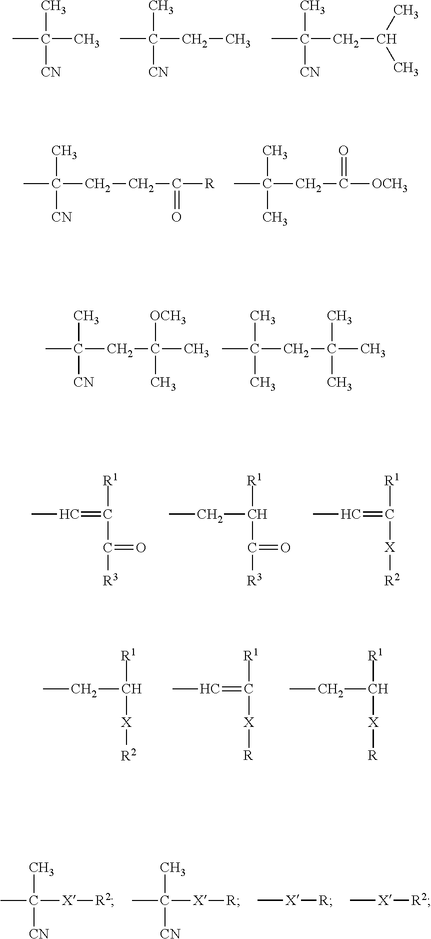

wherein R¹ is independently selected from H and CH₃;
wherein R² is defined above;
wherein R³ is selected from the group consisting of —NH—CH₂—CH(OH)—CH₃; —NH—CH₂CH₂—OH; —NH—CH₂CH₂CH₂—OH; —NH—C(CH₂OH)₃; —NH—CH(CH₂OH)₂; —NH—CH₂CH₂—N⁺(CH₃)₃Cl⁻; —O—CH₂CH₂—OH; —O—CH—(CH₂CH₂O)₂—H; —O—C—(CH₂CH₂O)₃—H; —O—CH₂CH₂—N⁺(CH₃)₃Cl⁻;
X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ—C(═O)—; wherein n is an integer from 1 to 7; o is an integer from 1 to 15; p, q and r are independently selected from the group consisting of 1, 2, and 3;
wherein if X is not a covalent bond, the group R² or R is attached via its end —S— or —NH— or —O— group or via a carbon (if R² is phenyl) to the end carbonyl group of the X linker;
   wherein the —CH₂— groups of X may be further independently substituted with one or more of the same or different side chains of a natural amino acid;
R is selected from the group consisting of —OH,

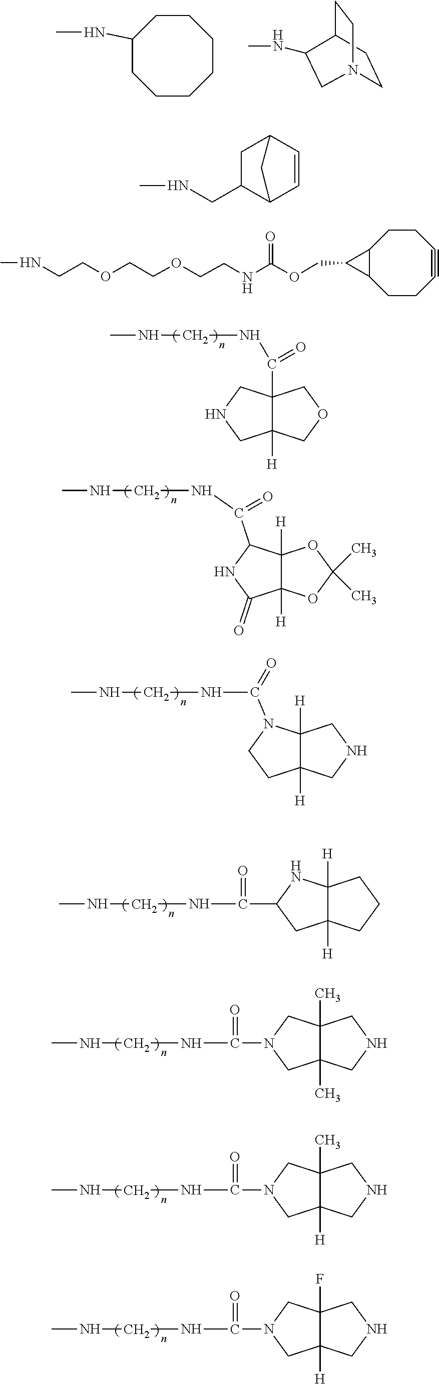

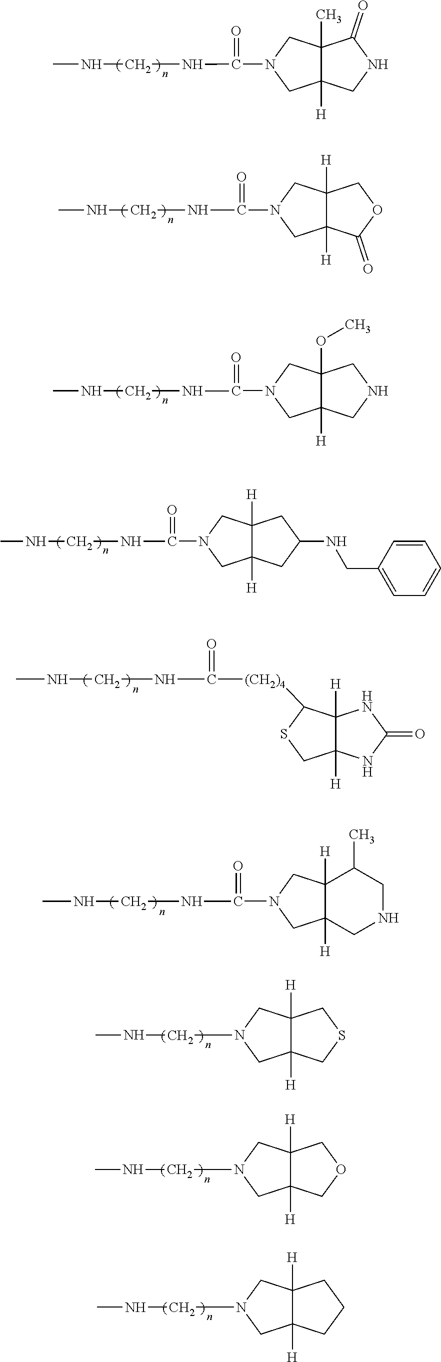

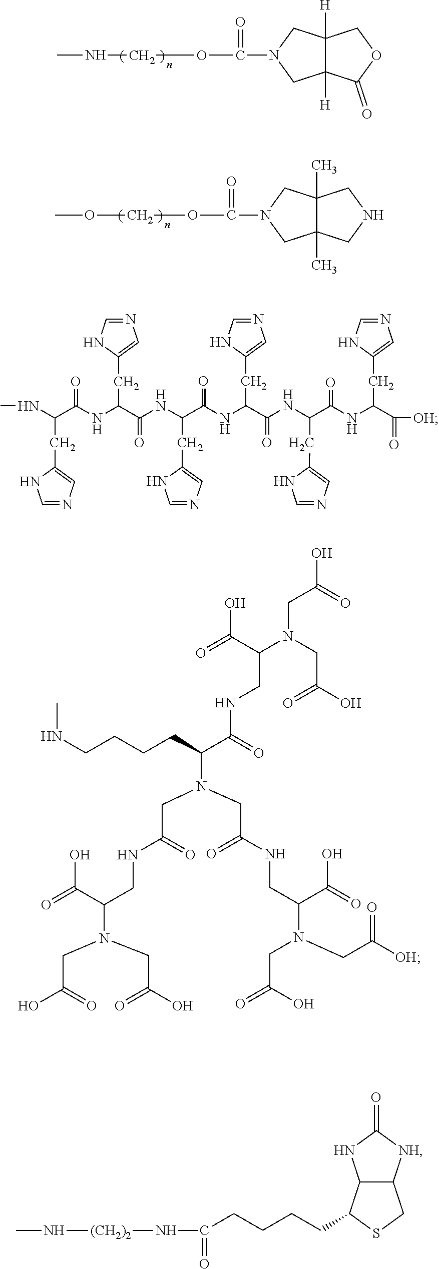

wherein n is an integer from 1 to 5;
and wherein the molecular weight of the polymer conjugate is in the range of from 5,000 to 500,000 g/mol, preferably in the range of from 10,000 to 150,000 g/mol, more preferably in the range of from 15,000 to 60,000 g/mol (corresponding to 80 to 400 monomer units).

Natural amino acids are histidine, isoleucine, leucine, lysine, methionine, phenylalanine, threonine, tryptophan, valine, arginine, cysteine, glutamine, glycine, proline, tyrosine, alanine, aspartic acid, asparagine, glutamic acid, serine, selenocysteine.

The side chains of a natural amino acid are the chains attached to the alpha-carbon of said amino acid.

In a preferred embodiment, the side chains of natural amino acids are selected from the group comprising methyl, isopropyl, isobutyl, —CH(CH₃)(CH₂CH₃), —CH₂OH, —CH(OH)(CH₃), —CH₂—(C₆H₄)OH, —(CH₂)₂—S—CH₃, —CH₂SH, —(CH₂)₄—NH₂, —CH₂COOH, —CH₂C(O)NH₂, —(CH₂)₂COOH, —(CH₂)₂C(O)NH₂, —(CH₂)₃NH—C(═NH)(NH₂), benzyl.

In a preferred embodiment, the X′ linker is selected from the group consisting of —NH—(CH₂)ₙ—C(═O)—; —(CH₂)ₚ—C(═O)—; covalent bond and —S—C(═S)—; wherein p and n are independently selected from the group consisting of 1, 2 and 3; preferably p and n are 2;
and R² is selected from the group consisting of —NH—(CH₂)_{b}—CH₃; —NH—(CH₂)_{b}—(CH═CH—CH₂)ₐ—(CH₂)_{b}—CH₃; —O—C(═O)—(CH₂)_{b}—CH₃; —S—(CH₂)_{b}—(CH═CH—CH₂)ₐ—(CH₂)_{b}—CH₃; —O—(CH₂)_{b}—CH₃; wherein a is an integer from 0 to 4, b is an integer from 4 to 17;
wherein the R² group is attached via its end —NH— or —O— group to the end carbonyl or thiocarbonyl group of the X′ linker.

In the most preferred embodiment, the X′ linker is selected from the group consisting of —S—C(═S)—; —(CH₂)ₚ—C(═O)—; —NH—(CH₂)ₙ—C(═O)—; wherein p and n are independently selected from the group consisting of 1, 2 and 3; preferably p and n are 2;
and R² is selected from the group consisting of —NH—(CH₂)₁₁—CH₃; —NH—(CH₂)₈—CH═CH—(CH₂)₇—CH₃; —O—(CH₂)₁₁—CH₃; wherein the R² group is attached via its end —NH— or —O— group to the end carbonyl or thiocarbonyl group of the X′ linker.

In a preferred embodiment, R is selected from the group consisting of

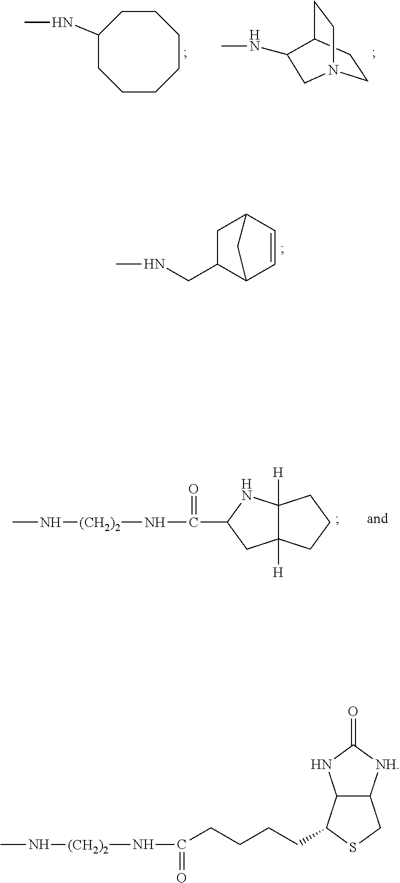

In one embodiment, the monomer units of the basic linear polymer are selected from monomer units of the general formula (K) and (L):

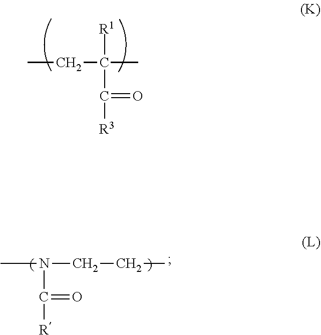

wherein R¹ and R³ are as defined above;
R′ is —CH₃ or —CH₂CH₃.

Thus, when the hydrophobically active anchor —X′—R² is attached via the X′ group as a side chain to the carbonyl group of the monomer unit of the basic linear polymer, it is meant that the —X′—R² group replaces the R³ and/or R′ groups in the above formulae (K) and (L).

In one embodiment, the hydrophobically active anchor in the polymer conjugate is attached as a side chain of a monomer unit of the basic linear polymer. In this embodiment, the polymer conjugate is a statistical linear copolymer of the general formula (XXX)

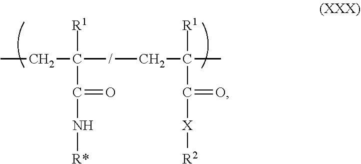

wherein
R* is —CH₂—CH(OH)—CH₃ or H; preferably, if R* is not H, then R¹ is CH₃;
the end groups are selected from the group consisting of —C(CH₃)₂—CN; —C(CH₃)(CH₂CH₃)—CN;

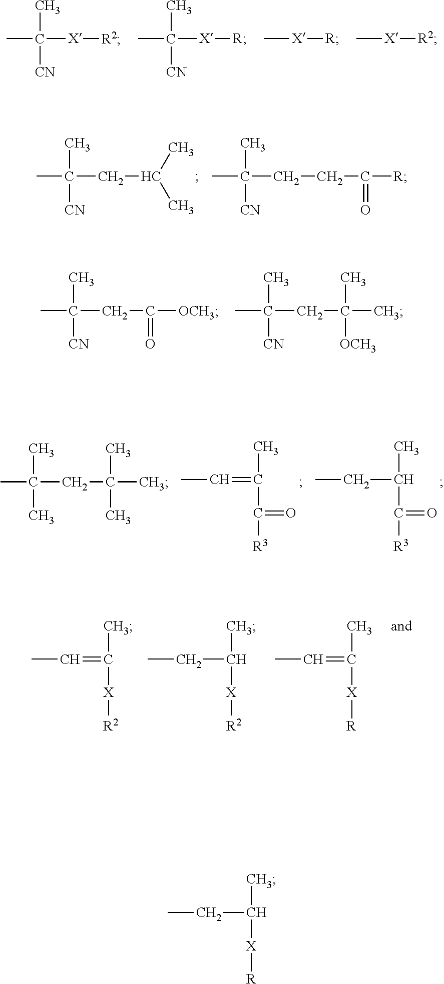

wherein the substituents X, X′, R, R¹, R² and R³ are as defined above. The content of the monomer units of the general formula (I)

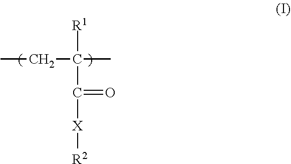

is in the range of from 0.5 to 10 mol %, based on the number of monomer units of the statistical linear copolymer.

Preferably X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ—C(═O)—; attached via its end —NH— or —O— group to the carbonyl group of the monomer unit of the general formula (I), wherein n is an integer from 1 to 7; o is an integer from 1 to 15; p, q and r are independently selected from the group consisting of 1, 2 and 3;
wherein the —CH₂— groups of X may be further independently substituted with one or more of the same or different side chains of a natural amino acid;
   with the proviso that if X is not a covalent bond, then R² group is attached via its end —NH— or —O— or —S— group or via the carbon atom of the phenyl group to the end carbonyl group of the X linker.

Preferably X′ is a linker selected from the group consisting of —NH—(CH₂)ₙ—C(═O)—; —(CH₂)ₚ—C(═O)—; a covalent bond; and —S—C(═S)—; wherein p and n are independently selected from the group consisting of 1, 2 and 3.

In a preferred embodiment, the end group is selected from the group consisting of

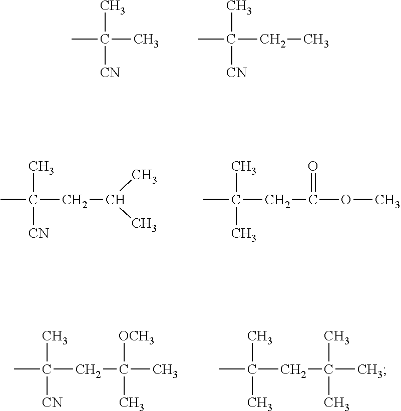

more preferably the end group is

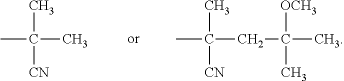

In a preferred embodiment X is —NH—(CH₂)ₙ—C(═O)—; wherein n is selected from the group consisting of 1, 2 and 3; more preferably n is 2;
and R² is selected from the group consisting of —NH—(CH₂)_{b}—CH₃; —NH—(CH₂)_{b}—(CH═CH—CH₂)ₐ—(CH₂)_{b}—CH₃; —O—(CH₂)_{b}—CH₃; wherein a is an integer from 0 to 4, b is an integer from 4 to 17;
wherein the R² group is attached via its end —NH— or —O— group to the end carbonyl group of the linker X.

In the most preferred embodiment X is —NH—(CH₂)ₙ—C(═O)—; wherein n is selected from the group consisting of 1, 2 and 3; more preferably n is 2;
and R² is selected from the group consisting of —NH—(CH₂)₁₁—CH₃; —NH—(CH₂)₈—CH═CH—(CH₂)₇—CH₃; —O—(CH₂)₁₁—CH₃; wherein the group R² is attached via its end —NH— or —O— group to the end carbonyl or thiocarbonyl group of the linker X.

Preferably, the polymer conjugate in this embodiment comprises from 0.7 to 5 mol % of monomer units of the general formula (I), more preferably from 0.7 to 3.5 mol % of monomer units of the general formula (I), even more preferably from 0.9 to 2 mol % of monomer units of the general formula (I).

In one embodiment, the monomer units of the basic linear polymer are monomer units of the general formula (K) as defined above, wherein from 0.5 to 10 mol % of the monomer units (K) of the basic linear polymer are statistically substituted with monomer units of the general formula (I) as defined above. Preferably, from 0.7 to 3.5 mol % of the monomer units (K) are replaced by monomer units of the general formula (I), more preferably from 0.9 to 2 mol % of the monomer units of the general formula (K) are replaced by monomer units of the general formula (I).

Depending on the method of preparation of said polymer conjugate, the polymer conjugate may contain active ester residues (for example, a thiazolidine-2-thione group) as side chains after attachment of the hydrophobically active anchor. These active groups are removed by reaction with an amino alcohol to give monomeric units of the general formula (III). In this embodiment, the polymer conjugate also contains, in addition to the monomer units of the general formula (I), monomer units of the general formula (III)

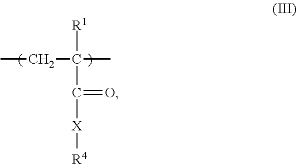

wherein R¹ is as defined above;
and R⁴ is selected from the group consisting of —NH—(CH₂)ₓ—CH₂(OH); —NH—(CH₂)_{y}—CH(OH)—CH₃; —NH—(CH₂)_{y}—CH(OH)—(CH₂)_{z}—CH₃; and —NH—C(═O)—CH₃; wherein x is an integer from 0 to 4, y is an integer from 0 to 3 and z is an integer from 1 to 4; with the proviso that if X is not a covalent bond, the R⁴ group is attached via its end —NH— group to the end carbonyl group of the linker X.

Thus, the polymer conjugate in this embodiment comprises from at least one monomer unit to 15 mol % of monomer units of the general formula (III), preferably from 1 to 12 mol % of monomer units of the general formula (III), more preferably from 5 to 9.5 mol % of monomer units of the general formula (III), based on the number of monomer units of the statistical linear copolymer.

In a preferred embodiment, the polymer conjugate may further comprise, in addition to the hydrophobically active anchor, an ‘interaction-reducing ligand’ enhancing the blocking activity of the entire blocker, and/or a bio-specific anchor. In both cases, these chains are attached to the polymer conjugate as side chains of monomer units. For example, biotin, His6 or tris-nitriloacetic acid may serve as a bio-specific anchor.

Examples of interaction-reducing ligands are aminocyclooctyl, aminoquinuclidin or norbornenyl.

In this embodiment, the polymer conjugate further contains from at least one monomer unit to 10 mol % of monomer units of the general formula (V), based on the number of monomer units of the statistical linear copolymer,

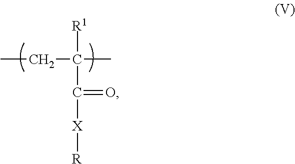

wherein
X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ—C(═O)—; wherein n is an integer from 1 to 7; o is an integer from 1 to 15; p, q and r are independently selected from the group consisting of 1, 2 and 3;
wherein the —CH₂— groups of X may further be independently substituted with one or more of the same or different side chains of the natural amino acid;
R is as defined above;
R¹ is H or CH₃;
with the proviso that if X is not a covalent bond, it is attached via its end —NH— or —O— group to the terminal carbonyl group of the monomeric unit of the general formula (V).

In one embodiment, the polymer conjugate is a statistical linear copolymer of the general formula (B)

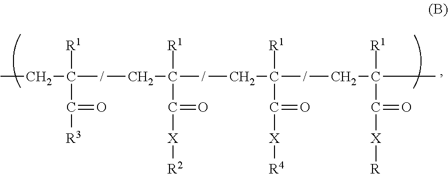

wherein
R¹ is H or CH₃;
R³ is selected from the group consisting of —NH—CH₂—CH(OH)—CH₃, —NH—CH₂CH₂—OH, —NH—CH₂CH₂CH₂—OH, —NHC(CH₂OH)₃, —NHCH(CH₂OH)₂, —NH—CH₂CH₂—N⁺(CH₃)₃Cl⁻, —O—CH₂CH₂—OH, —O—(CH₂CH₂O)₂—H; —O—(CH₂CH₂O)₃—H, —O—CH₂CH₂—N⁺(CH₃)₃Cl⁻;
X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ—C(═O)—; wherein n is an integer from 1 to 7; o is an integer from 1 to 15; p, q and r are independently selected from the group consisting of 1, 2 and 3; and wherein —CH₂— groups of X may be further independently substituted by one or more of the same or different side chains of a natural amino acid;
R² is as defined above;
R⁴ is as defined above;
R is as defined above;
with the proviso that if X is not a covalent bond, the groups R², R⁴ a R are bound via their end —S— or —NH— or —O— group or via a carbon atom (if R² is phenyl) to the terminal carbonyl group of the linker X;
and wherein the end groups of the statistical linear copolymer of the general formula (B) are as defined above. The monomer units in general formula (B) correspond to the monomer units of the general formula (K), (I), (III) and (V). Their content is preferably as follows: from 81 to 98.8 mol % of the monomer units of the general formula (K), from 0.7 to 3.5 mol % of the monomer units of the general formula (I), from 0.5 to 3.5 mol % of the monomer units of the general formula (V) and from at least one monomer unit to 12 mol % of the monomer units of the general formula (III).

In the most preferred embodiment of the polymer conjugate of the general formula (XXX), R¹ is methyl and R* is —CH₂—CH(OH)—CH₃.

In the most preferred embodiment of the polymer conjugate of the general formula B, R¹ is methyl and R³ is —NH—CH₂—CH(OH)—CH₃.

In one embodiment, the polymer conjugate comprises from 80 to 99 mol % of monomer units (K) of the basic linear polymer, preferably from 85 to 98 mol % of monomer units (K) of the basic linear polymer.

In one embodiment, the polymer conjugate comprises from 0.5 to 5 mol % monomer units of the general formula (I), preferably from 0.7 to 3.5 mol % monomer units of the general formula (I), more preferably from 0.9 to 2 mol % monomer units of the general formula (I).

In one embodiment, the polymer conjugate comprises from 0 to 15 mol % monomer units of the general formula (III), preferably from at least one monomer unit to 12 mol % monomer units of the general formula (III), more preferably from 1 to 9.5 mol % monomer units of the general formula (III).

In one embodiment, the polymer conjugate comprises from 0 to 10 mol % monomer units of the general formula (V), preferably from 0 to 5 mol % monomer units of the general formula (V), more preferably from 0.5 to 3.5 mol % monomer units of the general formula (V), more preferably from 0.7 to 2.5 mol % monomer units of the general formula (V).

In a preferred embodiment, the polymer conjugate comprises from 65 to 99.5 mol % monomer units of the basic linear polymer (monomer units of the general formula (K)), from 0.5 to 10 mol % monomer units of the general formula (I), from 0 to 10 mol % monomer units of the general formula (V), and from 0 to 15 mol % monomer units of the general formula (III).

Preferably, the polymer conjugate comprises from 78.0 to 99.3 mol % monomer units of the basic linear polymer, from 0.7 to 5 mol % monomer units of the general formula (I), from 0 to 5 mol % monomer units of the general formula (V), and from 0 to 12 mol % monomer units of the general formula (III).

Preferably, the polymer conjugate comprises from 81 to 98.8 mol % monomer units of the basic linear polymer, from 0.7 to 3.5 mol % monomer units of the general formula (I), from 0.5 to 3.5 mol % monomer units of the general formula (V), and from 0 to 12 mol % monomer units of the general formula (III).

In one embodiment, the polymer conjugate comprises from 98 to 99.1 mol % monomer units of the basic linear polymer, from 0.9 to 2 mol % monomer units of the general formula (I).

In one embodiment, wherein the hydrophobically active anchor in the polymer conjugate is attached as a side chain of a monomer unit of the basic linear polymer, the polymer conjugate is a statistical linear copolymer of the general formula (YYY)

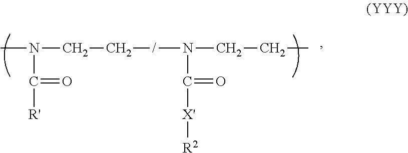

wherein R′ is —CH₃ or —CH₂CH₃;
X′ and R² are as defined above; preferably X′ is —(CH₂)ₚ—C(═O)—; wherein p is selected from the group consisting of 1, 2 and 3; preferably X′ is —(CH₂)₂—C(═O)—;
end groups are selected from the group consisting of —C(CH₃)₂—CN; —C(CH₃)(CH₂CH₃)—CN;

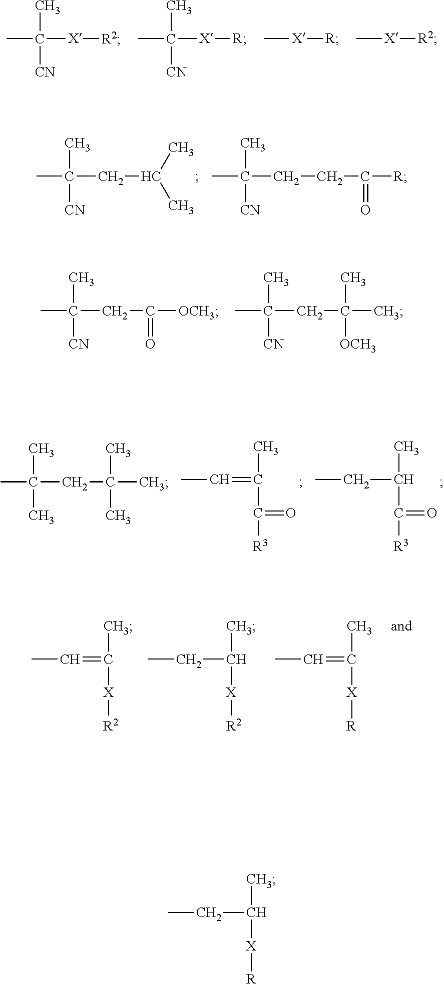

wherein substituents X, X′, R, R¹, R² and R³ are as defined above. The content of the monomer units of the general formula (II)

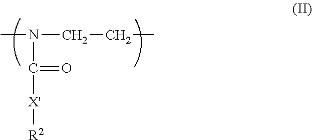

is in the range of from 0.5 to 10 mol %, based on the number of monomer units of the statistical linear copolymer.

Preferably X′ is X.

Preferably X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚC(═O)—; attached via its end —NH— or —O— group to the carbonyl group of the monomeric unit of the general formula (I), wherein n is an integer from 1 to 7; o is an integer from 1 to 15; p, q and r are independently selected from the group consisting of 1, 2 and 3;
wherein the —CH₂— groups of X may further be independently substituted with one or more of the same or different side chains of a natural amino acid;
   with the proviso that if X is not a covalent bond, then the R² group is attached via its end —NH— or —O— or —S— group or via the carbon atom of the phenyl group to the terminal carbonyl group of the linker X.

In a preferred embodiment X′ is —NH—(CH₂)ₙ—C(═O)—; wherein n is selected from the group consisting of 1, 2 and 3; more preferably n is 2;

and R² is selected from the group consisting of —NH—(CH₂)_{b}—CH₃; —NH—(CH₂)_{b}—(CH═CH—CH₂)ₐ—(CH₂)_{b}—CH₃; —O—(CH₂)_{b}—CH₃; wherein a is an integer from 0 to 4, b is an integer from 4 to 17;
wherein the R² group is attached via its end —NH— or —O— group to the terminal carbonyl group of the linker X′.

In the most preferred embodiment X′ is —NH—(CH₂)ₙ—C(═O)—; wherein n is selected from the group consisting of 1, 2 and 3; more preferably n is 2;

and R² is selected from the group consisting of —NH—(CH₂)₁₁—CH₃; —NH—(CH₂)₈—CH═CH—(CH₂)₇—CH₃; —O—(CH₂)₁₁—CH₃; wherein the R² group is attached via its end —NH— or —O— group to the terminal carbonyl or thiocarbonyl group of the linker X′.

Preferably, the polymer conjugate (YYY) in this embodiment comprises from 0.7 to 5 mol % monomer units of the general formula (II), more preferably from 0.7 to 3.5 mol % monomer units of the general formula (II), even more preferably from 0.9 to 2 mol % monomer units of the general formula (II).

In one embodiment, the monomer units of the basic linear polymer are monomer units of the general formula (L) as defined above, wherein from 0.5 to 10 mol % monomer units (L) of the basic linear polymer are statistically replaced by monomer units of the general formula (II) as defined above. Preferably from 0.7 to 3.5 mol % monomer units (L) are replaced by monomer units of the general formula (II), more preferably from 0.9 to 2 mol % monomer units of the general formula (L) are replaced by monomer units of the general formula (II).

Depending on the method of preparation of said polymer conjugate, the polymer conjugate may contain active ester residues (for example, a thiazolidine-2-thione group) as side chains after attachment of the hydrophobically active anchor. These active groups are removed by reaction with an amino alcohol to give monomer units of the general formula (IV). Thus, in this embodiment, the polymer conjugate further comprises, in addition to the monomer units of the general formula (II), monomer units of the general formula (IV)

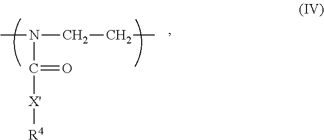

wherein X′ is as defined above; preferably X′ is —(CH₂)ₚ—C(═O)—; wherein p is selected from the group consisting of 1, 2 and 3; more preferably X′ is —(CH₂)₂—C(═O)—;
and R⁴ is selected from the group consisting of —NH—(CH₂)ₓ—CH₂(OH); —NH—(CH₂)_{y}—CH(OH)—CH₃; —NH—(CH₂)_{y}—CH(OH)—(CH₂)_{z}—CH₃; and —NH—C(═O)—CH₃; wherein x is an integer from 0 to 4, y is an integer from 0 to 3 and z is an integer from 1 to 4;
with the proviso that if X′ is not a covalent bond, then the R⁴ group is attached via its end —NH— group to the terminal carbonyl group of the linker X′.

Thus, the polymer conjugate in this embodiment comprises from at least one monomer unit to 15 mol % of monomer units, preferably from 1 to 12 mol % of monomer units, more preferably from 5 to 9.5 mol % monomer units of the general formula (IV), based on the number of monomer units of the statistical linear copolymer.

In a preferred embodiment, the polymer conjugate may further comprise, in addition to the hydrophobically active anchor, an ‘interaction-reducing ligand’ enhancing the blocking activity of the entire blocker, and/or a bio-specific anchor. In both cases, these chains are attached to the polymer conjugate as side chains of monomer units. For example, biotin, His6 or tris-nitriloacetic acid may serve as a bio-specific anchor. Examples of interaction-reducing ligands are aminocyclooctyl, aminoquinuclidin or norbornenyl.

In this embodiment, the polymer conjugate further contains from at least one monomer unit to 10 mol % of monomer units of the general formula (VI), based on the number of monomer units of the statistical linear copolymer,

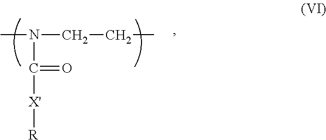

wherein
   X′ is selected from the group consisting of —(CH₂)ₚ—C(═O)—; covalent bond; —NH—(CH₂)ₙ—C(═O)—; —S—C(═S)—; NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ—C(═O)—; wherein n is an integer from 1 to 7; o is an integer from 1 to 15; p, q and r are independently selected from the group consisting of 1, 2 and 3; preferably X′ is —(CH₂)ₚ—C(═O)—; wherein p is selected from the group consisting of 1, 2 and 3; more preferably X′ is —(CH₂)₂—C(═O)—;
wherein the —CH₂— groups of X′ may further be independently substituted with one or more of the same or different side chains of the natural amino acid;
R is as defined above;
with the proviso that if X′ is not a covalent bond, the group X′ is bound via its end —NH— or —O— group to the carbonyl group of the monomer unit of the general formula (VI).

In another embodiment, the polymer conjugate is a statistical linear copolymer of the general formula (C)

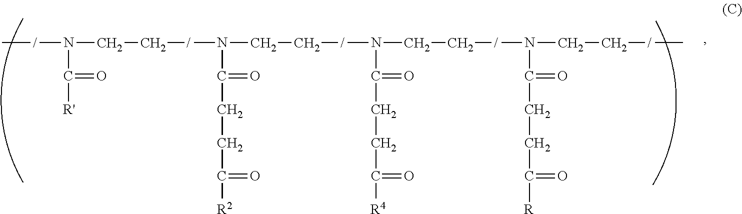

wherein
R′ is —CH₃ or —CH₂CH₃;
R² is as defined above;
R⁴ is as defined above;
R is as defined above;
wherein the end groups of the statistical linear copolymer of the general formula (C) are as defined above. The monomer units in the general formula (C) correspond to the monomer units of the general formula (L), (II), (IV) and (VI), present in the following amounts: from 81 to 98.8 mol % monomer units of the general formula (L), from 0.7 to 3.5 mol % monomer units of the general formula (II), from 0.5 to 3.5 mol % monomer units of the general formula (VI) and from at least one monomer unit to 12 mol % monomer units of the general formula (IV).

In one embodiment, the polymer conjugate comprises from 80 to 99 mol % monomer units of the basic linear polymer, preferably from 85 to 98 mol % of monomer units (L) of the basic linear polymer.

In one embodiment, the monomer units of the basic linear polymer are monomer units of the general formula (L) as defined above, wherein from 0.5 to 10 mol % of monomer units (L) of the basic linear polymer are statistically replaced by monomer units of the general formula (II) as defined above. Preferably from 0.7 to 3.5 mol % monomer units (L) are replaced by monomer units of the general formula (II), more preferably from 0.9 to 2 mol % monomer units of the general formula (L) are replaced by monomer units of the general formula (II).

In one embodiment, the polymer conjugate comprises from 0 to 15 mol % of monomer units of the general formula (IV), preferably from at least one monomer unit to 12 mol % monomer units of the general formula (IV), more preferably from 1 to 9.5 mol % monomer units of the general formula (IV).

In one embodiment, the polymer conjugate comprises from 0 to 10 mol % monomer units of the general formula (VI), preferably from 0 to 5 mol % monomer units of the general formula (VI), more preferably from 0.5 to 3.5 mol % monomer units of the general formula (VI), more preferably from 0.7 to 2.5 mol % monomer units of the general formula (VI).

In a preferred embodiment, the polymer conjugate comprises from 65 to 99.5 mol % monomer units of the basic linear polymer (monomer units of the general formula (L)), from 0.5 to 10 mol % monomer units of the general formula (II), from 0 to 10 mol % monomer units of the general formula (VI), and from 0 to 15 mol % monomer units of the general formula (IV).

Preferably, the polymer conjugate comprises from 78.0 to 99.3 mol % monomer units of the basic linear polymer, from 0.7 to 5 mol % monomer units of the general formula (II), from 0 to 5 mol % monomer units of the general formula (VI), and from 0 to 12 mol % monomer units of the general formula (IV).

Preferably, the polymer conjugate comprises from 81 to 98.8 mol % monomer units of the basic linear polymer, from 0.7 to 3.5 mol % monomer units of the general formula (II), from 0.5 to 3.5 mol % monomer units of the general formula (VI), and from 0 to 12 mol % monomer units of the general formula (IV).

In one embodiment, the polymer conjugate comprises from 98 to 99.1 mol % monomer units of the basic linear polymer, from 0.9 to 2 mol % monomer units of the general formula (II).

In one embodiment, the hydrophobically active anchor —X′—R² in the polymer conjugate is attached as at least one end group of the basic linear polymer.

In one preferred embodiment, wherein the hydrophobically active anchor —X′—R² in the polymer conjugate is bound as an end group of the basic linear polymer, the group —X′—R² is: —S—(CH₂)ₐ—CH—((CH₂)_{b}—CH₃)₂; —S—(CH₂)_{b}—(CH═CH—CH₂)ₐ—(CH₂)_{b}—CH₃; wherein a is an integer from 0 to 4, b is an integer from 4 to 17.

Most preferably the —X′—R² group is —S—C(═S)—S—(CH₂)₁₁—CH₃.

In one embodiment, the polymer conjugate comprises only monomer units of the general formula (K) or (L) while at least one end group is a group of general formula selected from

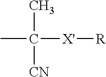

and —X′—R²; wherein X′ and R² are as defined above.

Preferably, in this embodiment X′ is selected from —S—C(═S)—; covalent bond; —(CH₂)ₚ—C(═O)—; wherein p is selected from the group consisting of 1, 2 and 3;

and R² is selected from —S—(CH₂)_{b}—(CH═CH—CH₂)ₐ—(CH₂)_{b}—CH₃; —O—C(═O)—(CH₂)_{b}—CH₃; —NH—C(═O)—(CH₂)_{b}—CH₃; wherein a is an integer from 0 to 4, b is an integer from 4 to 17. More preferably, the end group is —S—(CH₂)₁₁—CH₃ or —NH—(CH₂)₁₃—CH₃ or —O—C(═O)—(CH₂)₁₃—CH₃.

In the most preferred embodiment, at least one end group is selected from:

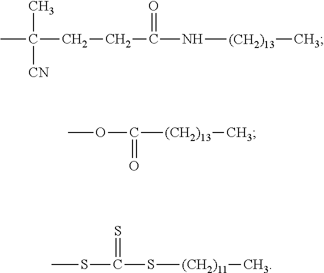

The remaining end group can be formed by a group

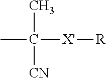

or —X′—R, wherein X′ and R are as defined above. Preferably, the remaining end group is, for example, the group

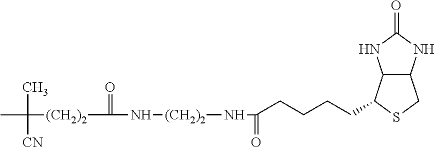

In one embodiment, a polyacrylamide, polymethacrylamide, polyacrylate, polymethacrylate, poly(N-(2-hydroxypropyl)methacrylamide) (monomer units of general formula (K)) is used as the basic linear polymer. In this embodiment, the polymer conjugate comprises exactly one hydrophobically active anchor which is bound to one end of the basic linear polymer via an X′ group, said polymer conjugate thus has the general formula (A)

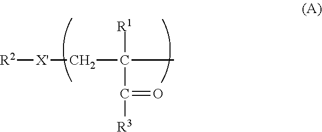

wherein
X′ is as defined above; preferably X′ is —S—C(═S)—;
R′ is H or CH₃;
R² is as defined above, preferably R² is —S—(CH₂)ₐ—C—((CH₂)_{b}—CH₃)₂ or —S—(CH₂)_{b}—(CH═CH—CH₂)ₐ—(CH₂)_{b}—CH₃; wherein a is an integer from 0 to 4, b is an integer from 4 to 17.
R³ is as defined above, preferably R³ is —NH—CH₂—CH(OH)—CH₃.

The second end group of the polymer conjugate of the general formula (A) is as defined above, preferably the second end group is

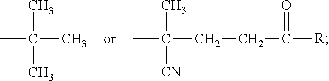

wherein R is as defined above. Most preferably, the second end group is

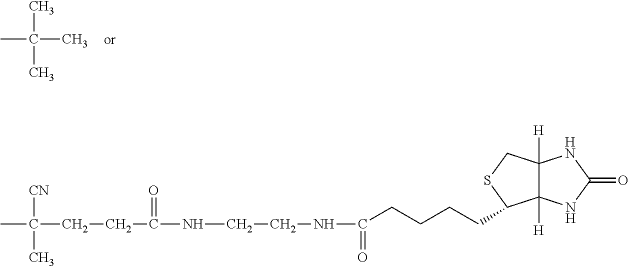

In an embodiment in which 2-polyoxazoline (monomer units of the general formula (L)) is used as the basic linear polymer, in one embodiment the polymer conjugate has the general formula (A′)

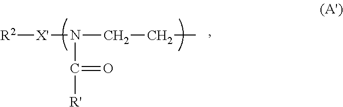

wherein R² and X′ are defined in the same way as in formula (A); R′ is —CH₃ or —CH₂CH₃. The end group of the polymer conjugate of general formula (A′) is defined as in formula (A).

In one embodiment, the hydrophobically active anchor —X′—R² in the polymer conjugate is attached to both ends of the basic linear polymer comprising monomer units (K) and/or (L). The X′ and R² groups are as defined as above. In this embodiment, the hydrophobically active anchor —X′—R² is preferably selected from the group comprising —S—(CH₂)ₐ—CH—((CH₂)_{b}—CH₃)₂ and —S—(CH₂)_{b}—(CH═CH—CH₂)ₐ—(CH₂)_{b}—CH₃; wherein a is an integer from 0 to 4, b is an integer from 4 to 17. Most preferably the —X′—R² group is —S—C(═S)—S—(CH₂)₁₁—CH₃.

Another object of the present invention is a method of preparing the polymer conjugate, said method comprising the following steps:
0) providing monomers of the linear copolymer;
i) polymerisation of the monomers of the linear copolymer, either by solution or precipitation radical polymerisation or RAFT polymerisation or ring-opening cationic polymerisation;
ii) binding of the hydrophobic anchor, optionally a hydrophobic-supporting anchor, to the linear copolymer to form a conjugate;
iii) optionally, binding of a bio-specific anchor.

Said polymer conjugates based on vinyl or cyclic monomers are prepared either by conventional radical solution or precipitation polymerisation or by controlled RAFT polymerisation or ring-opening cationic polymerisation.

The precursors of the polymer conjugates described above are preferably prepared by conventional radical polymerisation, both solution and precipitation, or by controlled RAFT polymerisation or ring-opening cationic polymerisation.

In the method of preparing the polymer conjugate of general formula (XXX), monomers of the general formulae (W), (Y), (Z) and (T) are used.

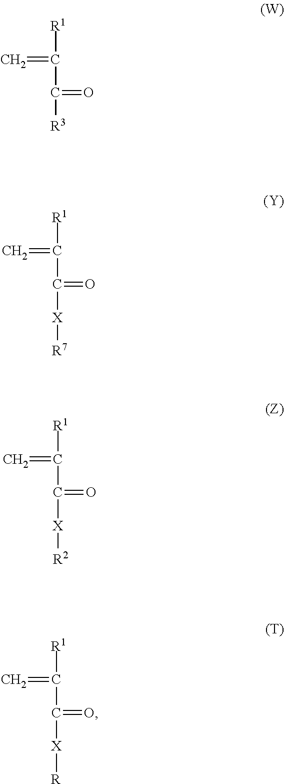

wherein R¹ is H or CH₃;
R³ is selected from the group consisting of —NH—CH₂—CH(OH)—CH₃, —NH—CH₂CH₂—OH, —NH—CH₂CH₂CH₂—OH, —NH—C—(CH₂OH)₃, —NH—CH—(CH₂OH)₂, —NH—CH₂CH₂—N⁺(CH₃)₃Cl⁻, —O—CH₂CH₂—OH, —O—CH—(CH₂CH₂O)₂—H; —O—C—(CH₂CH₂O)₃—H, —O—CH₂CH₂—N⁺(CH₃)₃Cl⁻;
R′ is —CH₃ or —CH₂CH₃ or —(CH₂)₂(C═O)OCH₃;
R² is selected from the group consisting of phenyl; —S—(CH₂)ₐ—CH—((CH₂)_{b}—CH₃)₂; —S—(CH₂)_{b}—(CH═CH—CH₂)ₐ—(CH₂)_{b}—CH₃; —NH—(CH₂)_{b}—CH₃; —NH—(CH₂)_{b}—(CH═CH—CH₂)ₐ—(CH₂)_{b}—CH₃; —O—(CH₂)_{b}—CH₃; —O—(CH₂)ₐ—C—((CH₂)_{b}—CH₃)₂; —NH—(CH₂)ₐ—C—((CH₂)_{b}—CH₃)₂; —O—(CH₂)_{b}—(CH═CH—CH₂)ₐ—(CH₂)_{b}—CH₃; and —O—C(═O)—(CH₂)ₐ—CH—((CH₂)_{b}—CH₃)₂; wherein a is an integer from 0 to 4, b is an integer from 4 to 17;
   with the proviso that if X is not a covalent bond, the group R² is attached via its end —S— or —NH— or —O— group or via carbon (in the case of phenyl) to the terminal carbonyl group of the linker X;
X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ—C(═O)—; attached via its end —NH— or —O— group to the carbonyl group of the monomer of the general formula (Y) or (Z) or (T), wherein n is an integer from 1 to 7; o is an integer from 1 to 15; p, q and r are independently selected from the group consisting of 1, 2 and 3;
   wherein the —CH₂— groups of X may further be independently substituted with one or more of the same or different side chains of a natural amino acid;
R⁷ is selected from

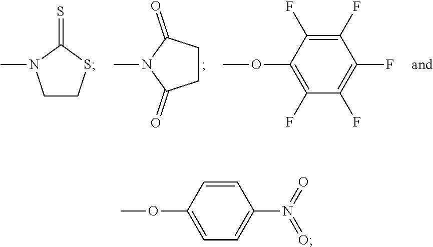

R is as defined above;
wherein step (i) is performed by solution or precipitation radical copolymerisation or RAFT polymerisation of the monomer (W) and of the monomers selected from the group comprising monomers of the general formula (Y), (Z) and (T);
wherein the total content of co-monomers of the general formulae (Y), (Z) and (T) is in the range of from 0.5 to 20 mol %, based on the total number of monomers; and wherein the reaction is carried out at a temperature in the range of from 30 to 100° C., preferably from 40 to 80° C., and in a solvent preferably selected from the group comprising dimethyl sulfoxide, N,N-dimethylacetamide, N,N-dimethylformamide, sulfolane, methanol, ethanol, dioxane, tetrahydrofuran, propanol, isopropanol, tert-butanol, N-vinylpyrrolidone, acetone, water, and aqueous buffers or mixtures thereof;
to form the linear copolymer.

Optionally, the R⁷ and/or thiazolidine-2-thione groups of the linear copolymer are subsequently conjugated to a hydrophobic molecule of the general formula R²—H, wherein R² is as defined above, to form a polymer conjugate comprising a hydrophobically active anchor attached via an amide or ester bond to the carbonyl group of the side chain of the monomer unit.

The R⁷ groups and/or thiazolidine-2-thione groups of the linear copolymer are optionally conjugated simultaneously with the preceding step or subsequently with not more than 10 mol % (0 to 10 mol %), based on the total number of monomers, of a bio-specific group of formula R—H, wherein R is as defined above, to form a polymer conjugate comprising a bio-specific group R attached via an amide or ester bond to the carbonyl group of the side chain of the monomer unit. Any unreacted reactive R⁷ groups and/or thiazolidine-2-thione groups are removed by reaction with aminoalcohol selected from the group comprising NH₂—(CH₂)ₓ—CH₂(OH); NH₂—(CH₂)_{y}—CH(OH)—CH₃; NH₂—(CH₂)_{y}—CH(OH)—(CH₂)_{z}—CH₃; wherein x is an integer from 0 to 4, y is an integer from 0 to 3 and z is from 1 to 4; preferably the aminoalcohol is 1-aminopropan-2-ol.

In one embodiment, the polymer conjugate according to the present invention is prepared by conventional radical polymerisation, both solution and precipitation, or by controlled RAFT polymerisation of monomers of the general formula (W) and (Z), wherein the content of monomers of the general formula (Z) in the reaction mixture is in the range of from 0.5 to 10 mol %, preferably from 0.5 to 5 mol %, of the total amount of monomers in the reaction mixture.

In one embodiment, the polymer conjugate according to the present invention is prepared by conventional radical polymerisation, both solution and precipitation, or by controlled RAFT polymerisation of monomers of the general formula (W), (Z) and (T), wherein the content of monomers of the general formula (Z) in the reaction mixture is in the range of from 0.5 to 10 mol % of the total amount of monomers in the reaction mixture, and the content of monomers of the general formula (T) in the reaction mixture is in the range of from 0.5 to 10 mol %, preferably from 0.5 to 5 mol %, of the total amount of monomers in the reaction mixture.

In one embodiment, the polymer conjugate according to the present invention is prepared by conventional radical polymerisation, both solution and precipitation, or by controlled RAFT polymerisation of monomers of the general formula (W) and (Y), wherein the content of monomers of the general formula (Y) in the reaction mixture is in the range of from 0.5 to 20 mol % of the total monomers in the reaction mixture. The reactive R⁷ groups of the (Y) monomer allow for the attachment of functional molecules (hydrophobically active anchors, bio-specific molecules, interaction-reducing ligands). The functional molecules are attached by covalent amide or ester bond, which is formed by the reaction of the reactive R⁷ groups introduced into the polymers with the amino or hydroxy groups of the functional molecules. As a solvent, DMA, DMF, DMSO or methanol is preferably used.

In one embodiment, the polymer conjugate of the general formula (A) according to the present invention is prepared by a method comprising the following steps:
(i) polymerising monomers of the general formula (W) as defined above, wherein step (i) is carried out by RAFT polymerisation at a temperature in the range of from 30 to 100° C., preferably from 40 to 80° C., and in a solvent preferably selected from the group comprising dimethyl sulfoxide, N,N-dimethylacetamide, N,N-dimethylformamide, sulfolane, methanol, ethanol, dioxane, tetrahydrofuran, propanol, isopropanol, tert-butanol, N-vinylpyrrolidone, acetone, water and aqueous buffers or mixtures thereof;
in the presence of a transfer agent of the general formula R⁸—X″-E,
wherein R⁸ is phenyl, —S—(CH₂)ₐ—CH—((CH₂)_{b}—CH₃)₂; —S—(CH₂)_{b}—(CH═CH—CH₂)ₐ—(CH₂)_{b}—CH₃; —S—(CH₂)_{b}—CH₃; wherein a is an integer from 0 to 4, b is an integer from 4 to 17;
X″ is —S—C(═S)—; wherein the R⁸ group is attached via its end —S— group or via carbon (in the case of phenyl) to the thiocarbonyl group of the linker X″; and
E is an end group selected from:

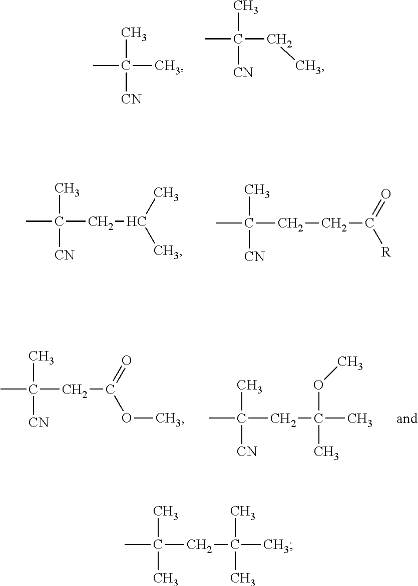

wherein R is as defined above, preferably R is selected from the group consisting of OH,

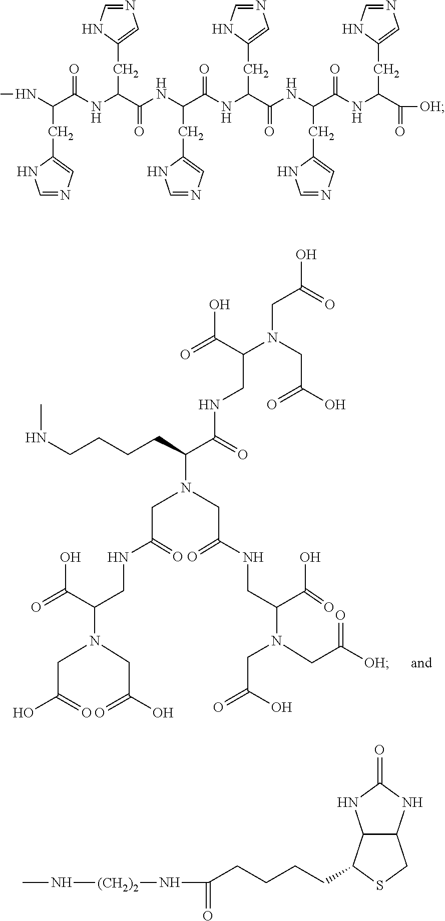

The transfer agent R⁸—X″-E is preferably 2-cyanoprop-2-yl-dodecyltrithiocarbonate or N-[2-[5-[(3aR,4R,6aS)-2-oxo-1,3,3a,4,6,6a-hexahydrothieno[3,4-d]imidazol-4-yl]pentanoylamino]ethyl]-4-cyano-4-dodecylsulfanylcarbothioylsulfanyl-pentanamide.
In the method of preparation of a polymer conjugate of the general formula (YYY), monomers of general formula (X) are used

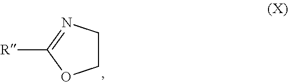

wherein
R″ is —CH₃ or —CH₂CH₃ or —(CH₂)₂(C═O)OCH₃;
   wherein step (i) is carried out by ring-opening cationic polymerisation; wherein one of the monomers is the monomer of the general formula (X), wherein R″ is —(CH₂)₂(C═O)OCH₃, and the total content of the monomer of the general formula (X), wherein R″ is —(CH₂)₂(C═O)OCH₃, is in the range of from 0.5 to 20 mol %, based on the total number of monomers; and wherein the reaction is carried out at a temperature in the range of from 30 to 100° C., and in a solvent preferably selected from the group comprising dimethyl sulfoxide, N,N-dimethylacetamide, N,N-dimethylformamide, sulfolane, methanol, ethanol, dioxane, tetrahydrofuran, propanol, isopropanol, tert-butanol, N-vinylpyrrolidone, acetone, water, and aqueous buffers or mixtures thereof;
to form a linear copolymer;
(ii) the step of hydrolysis of the methyl ester group of the substituent R″ wherein R″ is —(CH₂)₂(C═O)OCH₃; and subsequent reaction with thiazolidine-2-thione to form a reactive side chain containing a thiazolidine-2-thione group;
(iii) conjugation of the thiazolidine-2-thione group of the linear copolymer with a hydrophobic molecule of the general formula R²—H, wherein R² is as defined above, to form a polymer conjugate containing a hydrophobically active anchor attached by an amide or ester bond to the carbonyl group of the side chain of the monomer unit;
(iv) optionally, concurrently with or subsequent to the preceding step, conjugation of the thiazolidine-2-thione group of the linear copolymer of step (ii) with not more than 10 mol % (0 to 10 mol %) of a bio-specific group of formula R—H, based on the total number of monomers, wherein R is as defined above, to form a polymer conjugate comprising the bio-specific group R attached by an amide or ester bond to the carbonyl group of the side chain of the monomer unit;
(v) any unreacted reactive thiazolidine-2-thione groups are removed by reaction with an amino alcohol selected from the group comprising NH₂—(CH₂)ₓ—CH₂(OH); NH₂—(CH₂)_{y}—CH(OH)—CH₃; NH₂—(CH₂)_{y}—CH(OH)—(CH₂)_{z}—CH₃; wherein x is an integer from 0 to 4, y is an integer from 0 to 3 and z is from 1 to 4; preferably the aminoalcohol is 1-aminopropan-2-ol.

In one embodiment, the polymer conjugate according to the present invention is prepared by cationic polymerisation to open a ring of monomers of the general formula (X), wherein R″ is at one monomer —CH₃ or —CH₂CH₃ and for the other monomer R″ is —(CH₂)₂(C═O)OCH₃, wherein the content of monomers of the general formula (X), wherein R″ is —(CH₂)₂(C═O)OCH₃ in the reaction mixture, is in the range of from 0.5 to 10 mol % of the total amount of monomers in the reaction mixture.

In one embodiment, the polymer conjugate of the general formula (A′) is prepared by a method comprising the step of polymerisation of monomers of the general formula (X), wherein R′ is —CH₃ or —CH₂CH₃;
wherein the reaction is carried out by ring-opening cationic polymerisation at a temperature in the range of from 30 to 100° C., and in a solvent preferably selected from the group comprising dimethyl sulfoxide, N,N-dimethylacetamide, N,N-dimethylformamide, sulfolane, methanol, ethanol, dioxane, tetrahydrofuran, propanol, isopropanol, tert-butanol, N-vinylpyrrolidone, acetone, water and aqueous buffers or mixtures thereof;
under initiation with an initiator selected from methyl tosylate or 2-phenyl-2-oxazolinium tetrafluoroborate;
to form the polymer conjugate of the general formula (A′).

N-(2-hydroxypropyl)methacrylamide (HPMA) and other acrylamide, methacrylamide, acrylate and methacrylate type monomers (monomers of the general formula (W)) are commercially available.

Compounds of formulae (Y) and (Z) were prepared according to the procedures given in the literature (Subr, V. and K. Ulbrich, Synthesis and properties of new N-(2-hydroxypropyl)methacrylamide copolymers containing thiazolidine-2-thione reactive groups. React. Funct. Polym., 2006. 66: p. 1525-1538; Ulbrich, K., et al., Targeted Drug Delivery with Polymers and Magnetic Nanoparticles: Covalent and Noncovalent Approaches, Release Control, and Clinical Studies. Chemical Reviews, 2016. 116(9): p. 5338-5431). Compounds of formula (X) were prepared according to the procedures described in the literature (Glassner, M., M. Vergaelen and R. Hoogenboom, Poly(2-oxazoline)s: A comprehensive overview of polymer structures and their physical properties. Polymer International, 2018, 67(1), 32-45).

Compounds of formula (T) were obtained by a method wherein 3-methacrylamidopropanoic acid (Ma-β-Ala-OH) and an amine of the general formula NH₂—R (wherein R is as defined above) were dissolved in dichloromethane and a catalytic amount of 4-dimethylaminopyridine (DMAP) and successively N-(3-dimethylaminopropyl)-N′-ethylcarbodiimide hydrochloride (EDC·HCl) were added to the solution. The reaction mixture was stirred for 4 hours at laboratory temperature. The reaction mixture is then diluted with dichloromethane (DCM) and extracted with distilled water. The organic phase is dried with Na₂SO₄, concentrated under vacuum and crystallised in a freezer. The resulting crystals of compound of formula (T) are filtered off, washed with cold CHCl₃ and dried under vacuum.

The classical solution or precipitation polymerisation is initiated with the initiator 2,2-azobis(2-methylpropionitrile) ABIN or 2,2′-azobis[N-(2-carboxyethyl)-2-methylpropionamidine](V-70). The linear copolymer from step (i) is then terminated with a residue from the radical formed by decomposition of the initiator used.

The RAFT polymerisation is initiated by an initiator, preferably selected from the group consisting of V-70, AIBN, ABIK and ABIK-Biotin, wherein ABIK-Biotin is N-[2-[5-[(3aS,4S,6aR)-2-oxo-1,3,3a,4,6,6a-hexahydrothieno[3,4-d]imidazol-4-yl]pentanoylamino]ethyl]-4-[(E)-[4-[2-[5-[(3aS,4S,6aR)-2-oxo-1,3,3a,4,6,6a-hexahydrothieno[3,4-d]imidazol-4-yl]pentanoylamino]ethylamino]-1-cyano-1-methyl-4-oxo-butyl]azo]-4-cyano-pentanamid, in the presence of a chain transfer agent, preferably CTA-dodecylamine-biotin or CTA-dodecyl-COA, wherein CTA-dodecylamine-biotin is N-[2-[5-[(3aS,4S,6aR)-2-oxo-1,3,3a,4,6,6a-hexahydrothieno[3,4-d]imidazol-4-yl]pentanoylamino]ethyl]-4-cyano-4-dodecylsulfanylcarbothioylsulfanyl-pentanamide and CTA-dodecylamine-COA is 4-cyano-N-cyclooctyl-4-dodecylsulfanylcarbothioylsulfanyl-pentanamide.

In one example, a telechelic polymer is prepared by RAFT polymerisation that already contains a hydrophobically active anchor at the alpha-terminus and either an interaction-reducing ligand or a bio-specific anchor at the omega-terminus.

The attachment of the hydrophobic anchor to the linear copolymer from step (i) to form a polymer conjugate is accomplished by conjugation of reactive groups on the polymer, e.g. R⁷ or thiazolidine-2-thione (TT) groups of the monomer units after incorporation of a monomer of the general formula (Y), described above, with a hydrophobically active anchor (a precursor of the general formula R²—H, where R² is as defined above, such as a low molecular weight hydrophobic aliphatic chain) containing suitable reactive groups (e.g. an amine or hydroxyl group or SH group); wherein a low molecular weight aliphatic hydrophobic chain is a chain having a molecular weight in the range of from 100 to 400 g/mol. The hydrophobic anchor is thus attached to the linear copolymer by an amide or ester bond between the carbonyl group of the monomer unit (Y) and the —NH— or —O— group of X, formed by the reaction of reactive groups on the polymer, activated carboxylic groups with the amino or hydroxy groups of the hydrophobic anchor chain.

Any unreacted reactive groups of the copolymer, e.g. TT groups, may be removed by reaction with an amino alcohol selected from the group comprising NH₂—(CH₂)ₐ—CH₂(OH); NH₂—(CH₂)_{b}—CH(OH)—CH₃; NH₂—(CH₂)_{b}—CH(OH)—(CH₂)_{c}—CH₃; wherein a is an integer from 0 to 4, b is an integer from 0 to 3 and c is from 1 to 4; preferably with 1-aminopropan-2-ol, and/or the unreacted NH₂ groups can be removed by reaction with acetythiazolidin-2-thione.

This may be followed by the optional step of purification by column chromatography and lyophilisation of the final product from the previous step.

Any interaction-reducing ligand is attached analogously.

Optional modification of the macromolecular blocker with a bio-specific molecule allows selective interaction with some components of the immunoassay system and their attachment to the solid phase. The attachment of the bio-specific anchor to the linear copolymer is accomplished by conjugating reactive groups on the polymer, e.g., thiazolidine-2-thione (TT) groups of the monomeric units after incorporation of a monomer of the general formula (Y), described above, with a bio-specific anchor (e.g. a biotin derivative) containing suitable reactive groups (e.g. an amine or hydroxyl group); wherein the anchor is a molecule having a molecular weight in the range of from 80 to 1,000 g/mol. The anchor molecule, if any, is attached to the linear copolymer by an amide or ester bond formed between the reactive groups of the linear copolymer and the bound anchor, such as activated carboxylic groups, and the amino or hydroxyl groups of the bio-specific anchors.

In one variant, the synthetic macromolecular blocker is a telechelic vinyl copolymer in which functional moieties, a hydrophobic anchor, an interaction-reducing ligand, and/or a bio-specific anchor are attached to said vinylic synthetic homopolymer at both ends of its main polymer chain. In one embodiment, a monomer unit of the general formula (K) is used as the basic linear polymer and thus forms a polymer conjugate of the general formula (A″)

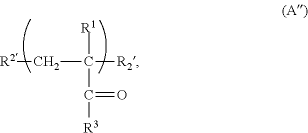

wherein R¹ and R³ are as defined above;
R²′ is independently selected from the group consisting of

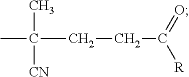

—S—C(═S)—C₆H₅, —S—C(═S)—S—(CH₂)ₐ—CH—((CH₂)_{b}—CH₃)₂; —S—C(═S)—S—(CH₂)_{b}—(CH═CH—CH₂)ₐ—(CH₂)_{b}—CH₃; —S—C(═S)—S—(CH₂)_{b}—CH₃; wherein a is an integer from 0 to 4, b is an integer from 4 to 17;
and R is as defined above, preferably R is

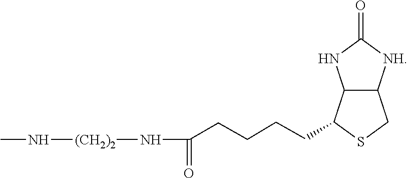

Thus, telechelic polymers of the general formula (A″) are composed only of the monomer units (K) defined above, and the ends of the main chains are terminated by groups R^{2′},
wherein at least one end of the main chain is terminated by the group —S—C(═S)—S—(CH₂)ₐ—C—((CH₂)_{b}—CH₃)₂ or —S—C(═S)—S—(CH₂)ₐ—(CH═CH—CH₂)ₐ—(CH₂)_{b}—CH₃ or —S—(CH₂)_{b}—CH₃; wherein a is an integer from 0 to 4, b is an integer from 4 to 17.

The attachment of functional molecules is in all cases realised by covalent bonds between reactive groups on functional molecules with reactive groups on copolymers or homopolymers. Preferably, the reaction of amino or hydroxyl groups localised on the functional molecules with activated carboxyl groups on the copolymers is utilised.

In one embodiment, the polymer conjugate is a 2-oxazoline based copolymer. The water-soluble copolymer based on poly(2-methyl-2-oxazoline) or poly(2-ethyl-2-oxazoline) (monomer units (X)) is prepared by ring-opening cationic polymerisation. The polymerisation is initiated by an initiator selected from: methyl tosilate or 2-phenyl-2-oxazolinium tetrafluoroborate. A copolymer containing carboxylic groups suitable for attaching can be prepared either by direct copolymerisation with a monomer unit containing a carboxylic group or a carboxylic acid methyl ester (monomer unit (X), wherein R′ is —(CH₂)₂(C═O)OCH₃) or by acid hydrolysis of the homopolymer, whereby a portion of the monomeric oxazoline units is converted to ethyliminium units to which a carboxylic group is then introduced by a polymeranalogous reaction.

The carboxyl groups contained in the polymeric precursor are activated in a subsequent step to the aminoreactive groups, see above, e.g., TT introduced via a polymeranalogous reaction with thiazolidine-2-thione. The polymeric precursor thus activated is used in the next step for an aminolytic reaction with functional moieties of the general formula R²—H, and optionally R—H, wherein R and R² are as defined above. These functional molecules are attached to this synthetic copolymer in its side chains, see general formula (C) described above.

The main active group is the ‘hydrophobically active’ anchor (group R²), which provides the activity of the entire polymer conjugate as a blocker of non-specific interactions and a suitable material for saturation of solid-phase immunoassay surfaces. The activity of the polymer conjugate is further enhanced by the interaction-reducing ligand (group R), which synergistically enhances the blocking and saturation ability of the whole synthetic blocker of non-specific interactions together with the hydrophobic-active anchor.

Optionally, a ‘bio-specifically binding’ anchor (group R, containing biotin, tris-NTA) is also covalently attached to the copolymer, which allows the copolymer to be used as a bispecific agent with an adherent anchor function for immobilisation to solid phase immunoassays such as tubes, microtiter plate wells, paramagnetic particles, etc., while immobilising other molecules through binding to the ‘bio-specifically binding’ anchor. Compounds binding one of the commercially available protein (purification) tags, such as tris-nitrilotriacetic acid (tris-NTA) binding oligohistidine tags, may be selected as suitable biospecific binding groups. The binding group may be, for example, biotin, which, due to the very strong biotin-avidin/streptavidin/neutravidin interaction, allows the entire copolymer to be used subsequently for immobilisation of biomolecules such as specific antibodies or antigens of various M.W.

All functional molecules may be attached to the copolymer via linkers based on aliphatic amino acids with 2 to 7 CH₂ units, classical amino acids, oligopeptides, or oligoPEG. The coupling allows the restriction of steric hindrance of functional molecules so that they can interact appropriately with other molecules or the surface of the immunoassay plate. Preferably, the linker is selected from the group comprising linkers based on aliphatic amino acids and classical amino acids and oligopeptides.

Polymer conjugates according to the present invention (synthetic macromolecular blockers of non-specific interactions) have several advantages over currently used animal-based protein blockers. The preparation of the macromolecular blocker is relatively simple and based on a controlled polymerisation principle, and the preparation of the individual polymer precursors and final blockers is highly reproducible. It is a synthetic copolymer that is not of animal origin. Due to the method of preparation, it is a product with completely reproducible properties.

The blocker can be used primarily as a reductant of non-specific interactions of the analyte and other components forming the analytical system on the surface of the solid phase. The blocker is present in excess in the solution and interacts with the surface of the solid phase and other components in solution, thereby suppressing non-specific interactions of the analyte and other components which would lead to an effect on the test result. The blocker can be used in immunochemical methods of any principle using solid phase of any format (paramagnetic particles, microtiter plate wells, tubes, beads, etc.). In addition to be used as a blocker, the copolymer can also be used to specifically bind some component of an immunoassay system to the solid phase.

The macromolecular blockers are preferably usable in immunochemical or analogous methods, preferably selected from luminescence, chemiluminescence, fluorescence, radioactive, or enzymatic assays, or assays using colloidal metal labelling, preferably in the methods of Luminescence Immunoassay (LIA), Immunoluminometric Analysis (ILMA), Chemiluminescence Immunoassay (CLIA), Immunochemiluminometric Analysis (ICMA), Electrochemiluminescence Analysis (ECL), Fluorescence Immunoassay (FIA), Immunofluorometric Analysis (IFMA), Radioactive Immunoassay (RIA), Immunoradiometric Analysis (IRMA), Enzyme Immunoassay (EIA), Immunoenzymometric Analysis (IEMA), Flow Cytometry, Immunohistochemistry (IHC), or Western Blotting (WB).

Thus, another object of the present invention is the use of the polymer conjugate in immunochemical methods as a blocker of non-specific interactions of an analyte and other components of the analytical system with the solid phase and, where appropriate, to capture specific antibodies or antigens or other molecules on the surface of the solid phase, in particular in the aforementioned immunochemical methods.

Copolymers are designed as highly active synthetic macromolecular inhibitors of non-specific interactions in analytical determinations using any detection signal (radioactivity, colorimetry, fluorescence, luminescence, turbidimetry, etc.).

### SUMMARY

The macromolecular non-specific interaction blocker according to the present invention is a functional molecule that serves in assays as a blocker of non-specific interactions of the analyte and other components of the analytical system with the solid phase, thereby significantly reducing non-specific interactions in the assay. The blocker can also be used for targeted capture of specific antibodies or antigens or other molecules on the surface of the solid phase. The macromolecular blocker is of synthetic origin and its preparation is highly defined. The activity of the blocker is based on a combination of two anchors, wherein the basic hydrophobic anchor causes high affinity for the solid phase and limits hydrophobic non-specific interactions in the solution itself, and the interaction-limiting ligand adds a further functionality to the whole macromolecular blocker, which results in an increase in its blocking activity.

The present invention enables the replacement of animal-derived proteins with a synthetic macromolecular blocker that is not only more effective in blocking activity itself, but it is also defined in its structure, has high batch-to-batch reproducibility, and does not need to be tested for the presence of viruses and other pathogens.

The present invention makes it possible to improve the results and the course of determinations within the framework of immunoassays and similar diagnostic determinations. The macromolecular blocker within the assay both replaces the portion responsible for limiting non-specific interactions and allows for the anchoring of important molecules, such as specific antibodies, to the surface of the solid phase.

### DESCRIPTION OF DRAWINGS

FIG. 1: Comparison of results using the system with BSA and with conjugates 2 and 3 (A, left) and cut-out for small values (B, right).

### EXAMPLES

All chemicals used were from Sigma-Aldrich unless otherwise stated.

The monomer compounds N-(2-hydroxypropyl)methacrylamide (HPMA) and 3-(3-methacrylamido-propanoyl)thiazolidin-2-thione (Ma-β-Ala-TT) were prepared according to a published procedure (S̆ubr, V. and K. Ulbrich, Synthesis and properties of new N-(2-hydroxypropyl)methacrylamide copolymers containing thiazolidine-2-thione reactive groups. React. Funct. Polym., 2006. 66: p. 1525-1538; Chytil, P., et al., N-(2-Hydroxypropyl)methacrylamide-based polymer conjugates with pH-controlled activation of doxorubicin for cell-specific or passive tumour-targeting. Synthesis by RAFT polymerisation and physicochemical characterisation. Eur. J. Pharm. Sci., 2010. 41: p. 473-482).

### Example 1

### Preparation of N-[3-(dodecylamino)-3-oxo-propyl]-2-methyl-prop-2-enamide Monomer

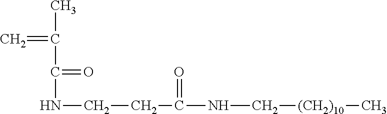

3-methacrylamidopropanoic acid (Ma-β-Ala-OH, 2 g) and NH₂-dodecylamine (2.54 g) were dissolved in 10 mL of dichloromethane (DCM) and a catalytic amount of 4-dimethylaminopyridine (DMAP) and successively 3.1 g of N-(3-dimethylaminopropyl)-N′-ethylcarbodiimide hydrochloride (EDC HCl) were added to the solution. The reaction mixture was stirred for 4 h at laboratory temperature. The reaction mixture was diluted with 10 mL of dichloromethane (DCM) and extracted with 3×10 mL of distilled water. The organic phase was dried with Na₂SO₄ and concentrated to 5 mL under vacuum and allowed to crystallise in a freezer. The precipitated crystals were filtered off, washed with cold CHCl₃ and dried under vacuum. 2.8 g of Ma-β-Ala-dodecylamine monomer was obtained. Characterisation by HPLC on a Chromolith High Resolution RP18e column showed a single peak with a retention time of 12.8 min at 220 nm.

### Example 2

### Preparation of N-[3-(octadecylamino)-3-oxo-propyl]-2-methyl-prop-2-enamide Monomer

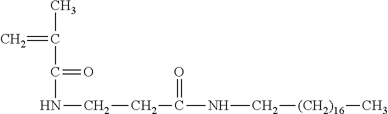

Ma-β-Ala-OH (2 g) and NH₂-octadecylamin (3.69 g) were dissolved in 15 mL dichloromethane (DCM) and a catalytic amount of 4-dimethylaminopyridine (DMAP) and 3.1 g EDC HCl were added to the solution successively. The reaction mixture was stirred for 4 h at laboratory temperature. The reaction mixture was diluted with 10 mL of DCM and extracted with 3×10 mL of distilled water. The organic phase was dried with Na₂SO₄ and concentrated to 5 mL under vacuum and allowed to crystallise in a freezer. The precipitated crystals were filtered off, washed with cold CHCl₃ and dried under vacuum. 3.8 g of Ma-β-Ala-octadecylamine monomer was obtained. Characterisation by HPLC on a Chromolith High Resolution RP18e column showed one peak with a retention time of 13.9 min at 220 nm.

### Example 3

### Preparation of a Statistical Poly(HPMA-co-Ma-β-Ala-TT) Copolymer by Radical Polymerisation

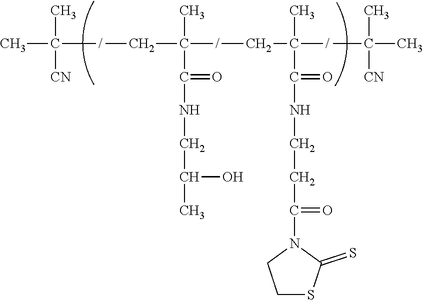

The copolymer containing thiazolin-2-thione (TT) reactive groups was prepared by solution radical copolymerisation of HPMA with 3-(3-methacrylamido-propanoyl)thiazolidine2-thione in DMSO at 60° C. for 6 hours. The total concentration of co-monomers in the polymerisation mixture was 12.5 wt %. and the concentration of AIBN (azobisisobutyronitrile) was 1.0 wt. %.

HPMA (1 g, 6.98 mmol), 3-(3-methakrylamidopropanoyl)thiazolidine2-thion (0.246 g, 0.95 mmol) and AIBN (100 mg) was dissolved in 7.8 mL DMSO. The solution was bubbled with argon for 10 min. Polymerisation was carried out in a sealed ampoule at 60° C. for 6 h. The copolymer was isolated by precipitation into a mixture of acetone-diethyl ether (3:1), filtered off, washed with acetone and diethyl ether and dried under vacuum. The polymer was dissolved in methanol (5.5 mL) and precipitated into a mixture of acetone-diethyl ether (3:1), filtered off, washed with acetone and diethyl ether and dried in vacuum. 0.965 mg of copolymer was obtained with a Mw of 37,700 and a dispersity of 2.04. The content of TT reactive groups was 12.8 mol %, based on the total number of monomer units.

### Example 4

### Preparation of Poly(HPMA-co-Ma-β-Ala-dodecylamine) Conjugate by Radical Precipitation Copolymerisation—Conjugate 1

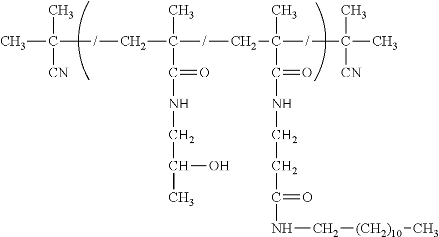

The poly(HPMA-co-Ma-β-Ala-dodecyl-amine) conjugate was prepared by precipitation radical copolymerisation of HPMA with N-[3-(dodecylamino)-3-oxo-propyl]-2-methyl-prop-2-enamide, prepared according to Example 1, in acetone at 60° C. for 6 hours. The total concentration of co-monomers in the polymerisation mixture was 12.5 wt % and the concentration of AIBN was 1.25 wt %.

HPMA (1 g, 6.98 mmol), N-[3-(dodecylamino)-3-oxo-propyl]-2-methyl-prop-2-enamide (0.046 g, 0.142 mmol) and AIBN (100 mg, 0.61 mmol) was dissolved in 8.8 mL acetone. The solution was bubbled with argon for 10 min. Polymerisation was carried out in a sealed ampoule at 60° C. for 6 h. The precipitated copolymer was filtered off, washed with acetone and diethyl ether and dried under vacuum. 0.879 g of copolymer (83.7%) was obtained with a molecular weight of Mw 46,300, a hydrophobic co-monomer unit content (derived from N-[3-(dodecylamino)-3-oxo-propyl]-2-methyl-prop-2-enamide) of 1.5 mol % and a dispersity of 1.63.

### Example 5

### Preparation of Poly(HPMA-co-Ma-β-Ala-dodecyl-amine) Conjugate (Conjugate 2)—Solution Polymerisation

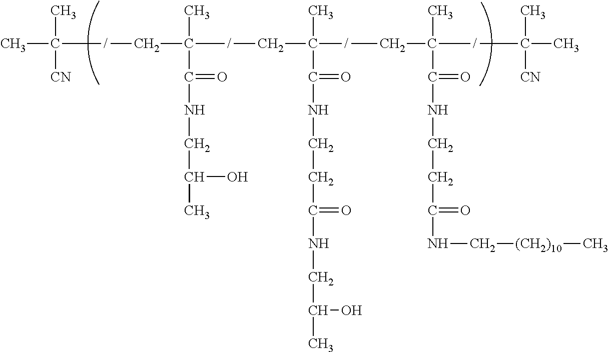

The polymer precursor poly(HPMA-co-Ma-β-Ala-TT) (0.2 g, Mw=37,700 g/mol, 12.8% mol TT) and dodecyl-amine (4.4 mg) were dissolved in 1.1 mL of DMSO. N,N-diisopropylethylamine (DIPEA) (10.4 μL) was then added and the reaction mixture was stirred for 4 h at laboratory temperature. Subsequently, 1-amino-propan-2-ol (10 μL) was added to the solution and the reaction mixture was stirred for 10 min. Then, the polymer conjugate poly(HPMA-co-Ma-β-Ala-dodecyl-amine) was isolated by precipitation into a mixture of acetone:diethyl ether (3:1), filtered off, washed with acetone and diethyl ether and dried under vacuum. The content of the hydrophobic co-monomer unit (derived from N-[3-(dodecylamino)-3-oxo-propyl]-2-methyl-prop-2-enamide) 0.8 mol % was determined in the sample hydrolysate (6N HCl, 115° C., 16 h) by HPLC with fluorescence detector (Ex. 229 nm, Em. 490 nm) on a Chromolith C18 column by pre-column derivatisation with o-naphthalenedialdehyde. The molecular weight of the conjugate Mw 34,300 and dispersity 1.67 were determined using a Shimadzu HPLC equipped with multi-angle scattering, viscometer and differential refractive index detectors in PBS buffer. The content of type (III) units was 11.2 mol %.

### Example 6

### Preparation of Poly(HPMA-co-Ma-β-Ala-dodecyl-amino-co-Ma-β-Ala-ED-biotin) Conjugate (Conjugate 3)—Solution Polymerisation

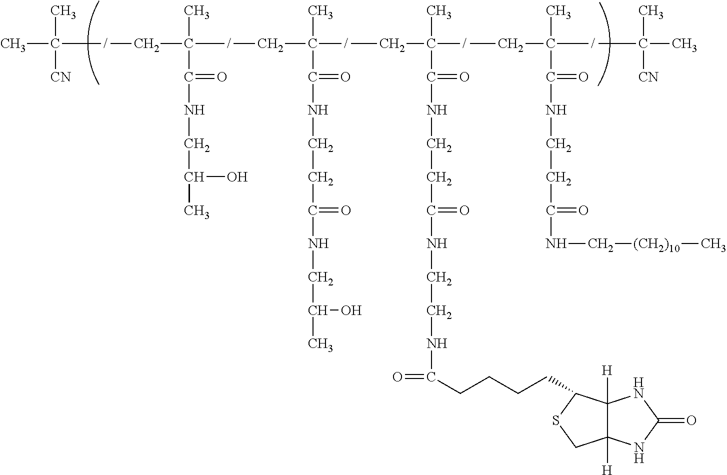

The polymer precursor poly(HPMA-co-Ma-β-Ala-TT) (0.5 g, Mw=41,600 g/mol, 11.8% mol TT) was dissolved in 2.8 mL of DMSO. To the solution was added 528 uL (14.5 mg) of a stock solution of dodecyl-amine (27.5 mg/l mL CHCl₃) and 489 uL (21.5 mg) of a stock solution of N-(2-aminoethyl) biotinamide trifluoroacetate (NH₂-ED-biotin. CF₃COOH) (22 mg/0.5 mL DMSO). N,N-diisopropylethylamine (DIPEA) (46.8 μL) was then added and the reaction mixture was stirred for 4 h at laboratory temperature. Subsequently, 1-amino-propan-2-ol (50 μL) was added to the solution and the reaction mixture was stirred for 10 min. Then, the C3 poly(HPMA-co-Ma-β-Ala-dodecyl-amino-co-Ma-β-Ala-ED-biotin) polymer conjugate was isolated by precipitation into a mixture of acetone:diethyl ether (3:1), filtered off, washed with acetone and diethyl ether, and dried under vacuum. The hydrophobic co-monomer unit (derived from N-[3-(dodecylamino)-3-oxo-propyl]-2-methyl-prop-2-enamide) content of 1 mol % was determined in the sample hydrolysate (6N HCl, 115° C., 16 h) by HPLC with fluorescence detector (Ex. 229 nm, Em. 490 nm) on a Chromolith C18 column by pre-column derivatisation with o-naphthalenedialdehyde, the content of biotin-containing units of 1.6 mol % was determined spectroscopically using the HABA/avidin kit, the content of type (III) units was 9.2 mol %. The molecular weight of the conjugate Mw 51,600 and dispersity 1.46 was determined using a Shimadzu HPLC equipped with multi-angle scattering, viscometer and differential refractive index detectors in PBS buffer.

### Example 7

### Preparation of Raft-poly(HPMA-co-Ma-β-Ala-TT) Statistical Copolymer by RAFT Polymerisation

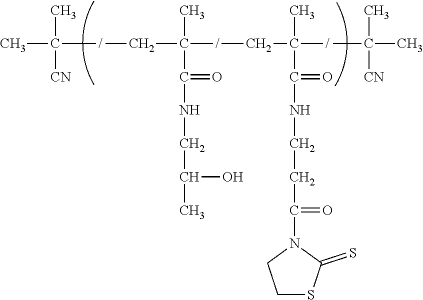

The polymer precursor Raft-poly(HPMA-co-Ma-β-Ala-TT) was prepared by RAFT (reversible addition-fragmentation chain-transfer) copolymerisation. 1.25 g of HPMA (88% mol) was dissolved in 10.9 mL of tert-butanol and 308 mg of Ma-β-Ala-TT (12% mol) dissolved in 2.8 mL of DMAc (dimethylacetamide), 3.13 mg of 2-ethylsulfanylcarbothioyl-2-methyl-propanenitrile and 2.35 mg of 2,2′-azobis(4-methoxy-2,4-dimethylvaleronitrile) was added to the solution and the solution was transferred to a polymerisation ampoule. The mixture was bubbled for 10 min with argon and then the ampoule was sealed. The polymerisation reaction was carried out at 40° C. (24 h). The polymer precursor Raft-poly(HPMA-co-Ma-β-Ala-TT) was isolated by precipitation into a mixture of acetone:diethyl ether (3:1), filtered off, washed with acetone and diethyl ether and dried under vacuum. The terminal trithiocarbonate groups were removed according to a previously published procedure Perrier, S., P. Takolpuckdee, and C. A. Mars, Reversible addition-fragmentation chain transfer polymerization: End group modification for functionalized polymers and chain transfer agent recovery. Macromolecules, 2005. 38(6): p. 2033-2036. The polymer precursor poly(HPMA-co-Ma-β-Ala-TT) with molecular weight Mw=49,000 g/mol, polydispersity D=1.23 and containing 11.6 mol % of reactive thiazolidine-2-thione (TT) groups, based on the total number of monomer units, was obtained by this procedure.

### Example 8

### Preparation of Raft-poly(HPMA-co-Ma-β-Ala-dodecyl-amine) Conjugate (Conjugate 4)

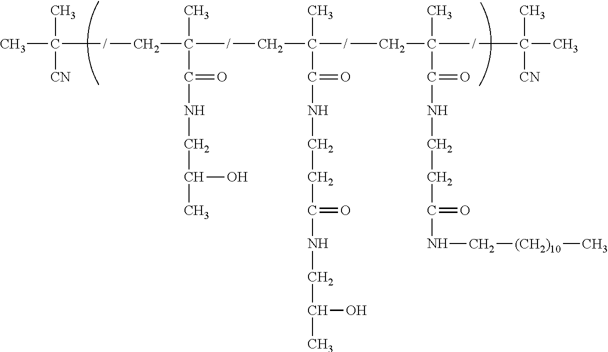

The polymer precursor Raft-poly(HPMA-co-Ma-β-Ala-TT) (82 mg, Mw=49,000 g/mol, 11.6% mol TT), prepared according to Example 7, and dodecyl-amine (1.8 mg) were dissolved in 0.35 mL of DMSO. N,N-diisopropylethylamine (DIPEA) (4.2 μL) was then added and the reaction mixture was stirred for 4 h at laboratory temperature. Subsequently, 1-amino-propan-2-ol (10 μL) was added to the solution and the reaction mixture was stirred for 10 min. Then, the polymer conjugate Raft-poly(HPMA-co-Ma-β-Ala-dodecyl-amine) was isolated by precipitation into a mixture of acetone:diethyl ether (3:1), filtered off, washed with acetone and diethyl ether, and dried under vacuum. The content of the hydrophobic co-monomer unit (derived from N-[3-(dodecylamino)-3-oxo-propyl]-2-methyl-prop-2-enamide) 0.6 mol % was determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 11 mol %. The molecular weight of the conjugate Mw 66,000 and the dispersity 1.09 were determined using a Shimadzu HPLC equipped with a multi-angle scattering, viscometer and RI detector.

### Example 9

### Preparation of Raft-poly(HPMA-co-Ma-β-Ala-dodecyl-amino-co-Ma-β-Ala-ED-biotin) (Conjugate 5)

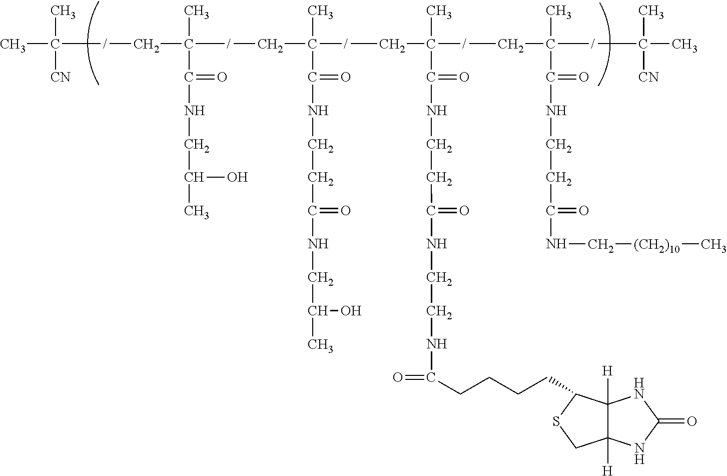

The polymer precursor Raft-poly(HPMA-co-Ma-β-Ala-TT) (50 mg, Mw=49,000 g/mol, 11.6% mol TT), prepared according to Example 7, dodecyl-amine (1.0 mg) and NH₂-ED-biotin·CF₃COOH (2.4 mg) was dissolved in 0.22 mL of DMSO. Then N,N-diisopropylethylamine (DIPEA) (9.9 μL) was added and the reaction mixture was stirred for 4 h at laboratory temperature. Subsequently, 1-amino-propan-2-ol (10 μL) was added to the solution and the reaction mixture was stirred for 10 min. Then, the polymer conjugate Raft-poly(HPMA-co-Ma-β-Ala-dodecyl-amino-co-Ma-β-Ala-ED-biotin) was isolated by precipitation into a mixture of acetone:diethyl ether (3:1), filtered off, washed with acetone and diethyl ether and dried under vacuum. The content of the hydrophobic co-monomer unit (derived from N-[3-(dodecylamino)-3-oxo-propyl]-2-methyl-prop-2-enamide) 0.9 mol % was determined in the hydrolysate of the sample (6 N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 8.7 mol %. The molecular weight of the conjugate Mw 59,000 and dispersity 1.18 were determined using a Shimadzu HPLC equipped with multi-angle scattering, viscometer and differential refractive index detectors on Superose 6increase in PBS buffer. The content of biotin-containing units of 2.0 mol % was determined using the HABA-Avidin kit from Sigma-Aldrich.

### Example 10

### Preparation of poly(HPMA-co-Ma-β-Ala-oleylamine) Conjugate—Conjugate 6

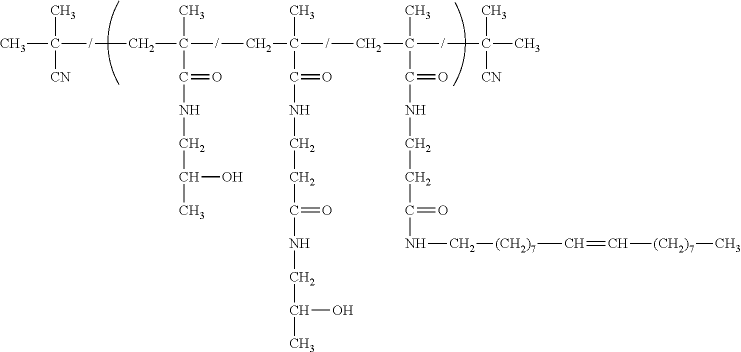

The polymer precursor poly(HPMA-co-Ma-β-Ala-TT) (0.2 g, Mw=37,700 g/mol, 12.8% mol TT), prepared according to Example 3, and oleylamine (4.0 mg) were dissolved in 1.1 mL of DMSO. N,N-diisopropylethylamine (DIPEA) (6.5 μL) was then added and the reaction mixture was stirred for 4 h at laboratory temperature. Subsequently, 1-amino-propan-2-ol (10 μL) was added to the solution and the reaction mixture was stirred for 10 min. Then the polymer conjugate poly(HPMA-co-Ma-β-Ala-oleylamine) was isolated by precipitation into a mixture of acetone:diethyl ether (3:1), filtered off, washed with acetone and diethyl ether and dried under vacuum. The content of the hydrophobic co-monomer unit (containing oleylamine) of 0.8 mol % was determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 12 mol %. The molecular weight of the conjugate Mw 45,200 and dispersity 1.70 were determined using a Shimadzu HPLC equipped with multi-angle scattering, viscometer and differential refractive index detectors in PBS buffer.

### Example 11

### Preparation of Poly(HPMA-co-Ma-β-Ala-stearylamine) Conjugate—Conjugate 7

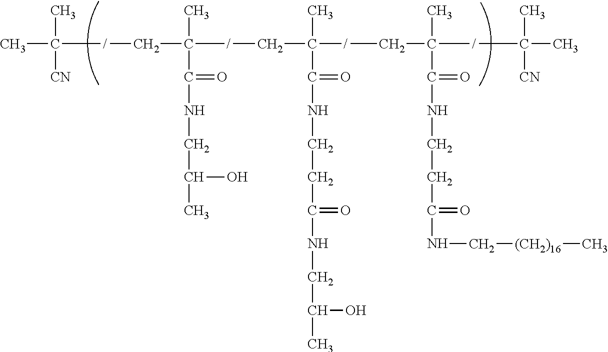

The polymer precursor poly(HPMA-co-Ma-β-Ala-TT) (0.2 g, Mw=37,700 g/mol, 12.8% mol TT), prepared according to Example 3, and stearylamine (3.6 mg) were dissolved in 1.1 mL of DMSO. N,N-diisopropylethylamine (DIPEA) (5.8 μL) was then added and the reaction mixture was stirred for 4 h at laboratory temperature. Subsequently, 1-amino-propan-2-ol (10 μL) was added to the solution and the reaction mixture was stirred for 10 min. Then, the polymer conjugate poly(HPMA-co-Ma-β-Ala-stearylamine) was isolated by precipitation into a mixture of acetone:diethyl ether (3:1), filtered off, washed with acetone and diethyl ether and dried under vacuum. The content of the hydrophobic co-monomer unit (containing stearylamine) of 0.9 mol % was determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 11.9 mol %. The molecular weight of the conjugate Mw 46,300 and the dispersity 1.68 were determined by Shimadzu HPLC equipped with multi-angle dispersion, viscometer and differential refractive index detectors in PBS buffer.

### Example 12

### Preparation of poly(HPMA-co-Ma-β-Ala-dodecanol) Conjugate—Conjugate 8

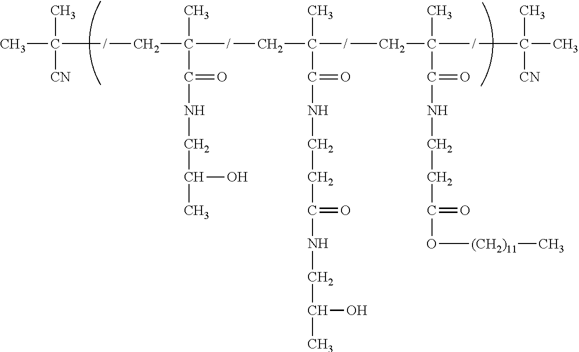

The polymer precursor poly(HPMA-co-Ma-β-Ala-TT) (200 mg, Mw=40,200 g/mol, 10.8% mol PFP) and 1-dodecanol (3.6 mg) were dissolved in 1.1 mL DMSO. N,N-diisopropylethylamine (DIPEA) (5.8 μL) was then added and the reaction mixture was stirred for 4 h at laboratory temperature. Subsequently, l-amino-propan-2-ol (10 μL) was added to the solution and the reaction mixture was stirred for 10 min. Then the polymer conjugate poly(HPMA-co-Ma-β-Ala-dodecanol) was isolated by precipitation into a mixture of acetone:diethyl ether (3:1), filtered off, washed with acetone and diethyl ether and dried under vacuum. The content of the hydrophobic co-monomer unit (containing dodecanol) of 1.1 mol % was determined by ¹H NMR spectroscopy. The content of the type (III) units was 9.7 mol %. The molecular weight of the conjugate Mw 43,400 and the dispersity 1.81 were determined using a Shimadzu HPLC equipped with multi-angle dispersion, viscometer and differential refractive index detectors in PBS buffer.

### Example 13

### Preparation of N-[2-[5-[(3aR,4R,6aS)—2-oxo-1,3,3a,4,6,6a-hexahydrothieno[3,4-d]imidazol-4-yl]pentanoylamino]ethyl]-4-cyano-4-dodecylsulfanylcarbothioylsulfanyl-pentanamide (dodecyl-trithio-biotin) Transfer Agent

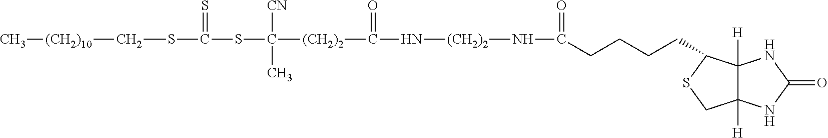

Dodecyl-trithio-biotin was prepared by a two-step synthesis. In the first step, 2-dodecylsulfanylcarbothioylsulfanyl-2-methyl-5-oxo-5-(2-thioxothiazolidin-3-yl)pentanenitrile (dodecyl-trithio-TT) was prepared by the reaction of 4-cyano-4-dodecylsulfanylcarbothioylsulfanyl-pentanoic acid (dodecyl-trithio-COOH) with thiazolidine-2-thione (TT) in dichloromethane in the presence of EDC·HCl. In the second step, the prepared dodecyl-trithio-TT was reacted with N-biotinyl-ethylenediamine trifluoroacetate in DMF to form the desired dodecyl-trithio-biotin.

Dodecyl-trithio-COOH (100 mg, 0.247 mmol) and thiazolidine-2-thione (31.0 mg, 0.260 mmol) were dissolved in 4 mL of dichloromethane and catalytic amounts of dimethylaminopyridine and EDC·HCl (57 mg, 0.297 mmol) were added to the solution. The reaction mixture was stirred for 4 h at laboratory temperature. The reaction mixture was washed with 2×10 mL distilled water and the DCM layer was dried with anhydrous Na₂SO₄ and the DCM was evaporated under vacuum. Dodecyl-trithio-TT (100 mg, 0.247 mmol) was dissolved in 1 mL DMF. NH₂-ED-biotin·CF₃COOH (99.15 mg, 0.247 mmol) was dissolved in 1 mL DMF and (43 uL, 0.247 mmol) DIPEA was added to the solution. The NH₂-ED-biotin solution was then added to the dodecyl-trithio-TT solution and stirred for 1 h at laboratory temperature. During the reaction, the initially yellowish solution turned colourless. The reaction mixture was poured into 100 mL of distilled water. The precipitated product was isolated by centrifugation. The product was dissolved in isopropanol and the isopropanol was evaporated in vacuum to give the solid product.

### Example 14

### Preparation of Poly(HPMA-co-Ma-β-Ala-dodecyl-amino-co-Ma-β-Ala-TT) Precursor and Poly(HPMA-co-Ma-β-Ala-dodecyl-amino-co-Ma-β-Ala-propanol) Conjugate (Conjugate 9) by Radical Terpolymerisation

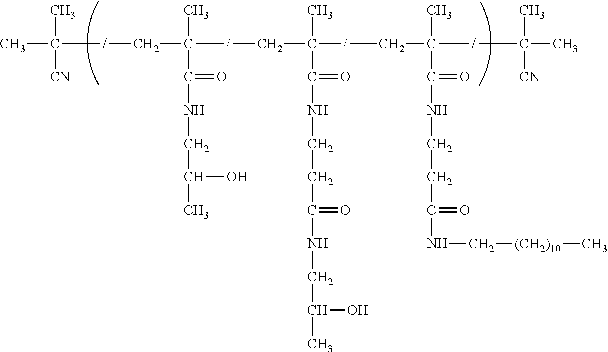

The poly(HPMA-co-Ma-β-Ala-dodecyl-amino-co-Ma-β-Ala-TT) conjugate was prepared by solution radical terpolymerisation of HPMA with N-[3-(dodecylamino)-3-oxo-propyl]-2-methyl-prop-2-enamide and 3-(3-methacrylamidopropanoyl)thiazolidin-2-thione in DMSO at 60° C. for 6 hours. The concentration of co-monomers in the polymerisation mixture was 12.5 wt %. and the concentration of AIBN was 1.25 wt %.

HPMA (1 g, 6.98 mmol), N-[3-(dodecylamino)-3-oxo-propyl]-2-methyl-prop-2-enamid (0,051 g, 0,157 mmol) a 3-(3-methacrylamidopropanoyl)thiazolidin-2-thione (0,182 g, 0,706 mmol) a AIBN (118 mg) was dissolved in 7.4 mL DMSO. The solution was bubbled with argon for 10 min. Polymerisation was carried out in a sealed ampoule at 60° C. for 6 h. The copolymer was isolated by precipitation into 200 mL of acetone-diethyl ether mixture (3:1), filtered off, washed with acetone and diethyl ether and dried under vacuum. 0.999 g of copolymer (84.5%) containing 10.2 mol % of reactive TT groups was obtained.

The polymer thus obtained (200 mg) was dissolved in DMSO (1.2 mL) and 20 uL of 1-amino-2-propanol was added to the solution and stirred for 10 min until the reaction mixture decolourised. Conjugate 9 was precipitated into 30 mL of acetone-diethyl ether mixture (3:1), filtered off, washed with acetone and diethyl ether and dried in vacuum. 184 mg of the copolymer was obtained with Mw 38,700 and a dispersity of 1.51. The content of the hydrophobic co-monomer unit (derived from N-[3-(dodecylamino)-3-oxo-propyl]-2-methyl-prop-2-enamide) was 0.8 mol %. The content of type (III) units was 9.4 mol %.

### Example 15

### Preparation of Poly(HPMA-co-Ma-β-Ala-dodecyl-amino-co-Ma-β-Ala-aminoquinuclidine) Polymer Conjugate (Conjugate 10) by Radical Ter-Polymerisation

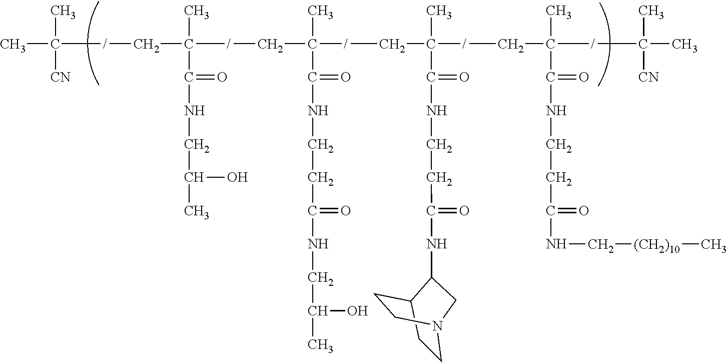

The polymer precursor poly(HPMA-co-Ma-β-Ala-TT) (0.2 g, Mw=41,200 g/mol, Ð=2.08, 11.8% mol TT), prepared according to Example 3, and dodecyl amine (4.4 mg) were dissolved in 1.0 mL of DMSO. N,N-diisopropylethylamine (DIPEA) (4.8 μL) was then added and the reaction mixture was stirred for 1 h at laboratory temperature. Subsequently, 55 μL of stock 3-aminoquinuclidine solution (12.41 mg/200 uL DMSO) and N,N-diisopropylethylamine (DIPEA) (10.0 μL) were added to the solution and the reaction mixture was stirred for 24 h at laboratory temperature. Then 1-amino-propan-2-ol (10 μL) was added to the solution and the reaction mixture was stirred for 10 min. Then the polymer conjugate 10, poly(HPMA-co-Ma-β-Ala-dodecyl-aminoquinuclidine), was isolated by precipitation into a mixture of acetone:diethyl ether (3:1), filtered off, washed with acetone and diethyl ether, and dried under vacuum. The content of the hydrophobic co-monomer unit (containing dodecylamine) 0.7 mol % and the content of the co-monomer unit containing aminoquinuclidine 0.9 mol % were determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 10.2 mol %. The molecular weight of the conjugate Mw 40,000 and dispersity 1.44 were determined using a Shimadzu HPLC equipped with multi-angle scattering, viscometer and differential refractive index detectors in PBS buffer.

### Example 16

### Preparation of Poly(HPMA-co-Ma-β-Ala-dodecyl-amino-co-Ma-β-Ala-aminocyclooctane) Polymer Conjugate (Conjugate 11)

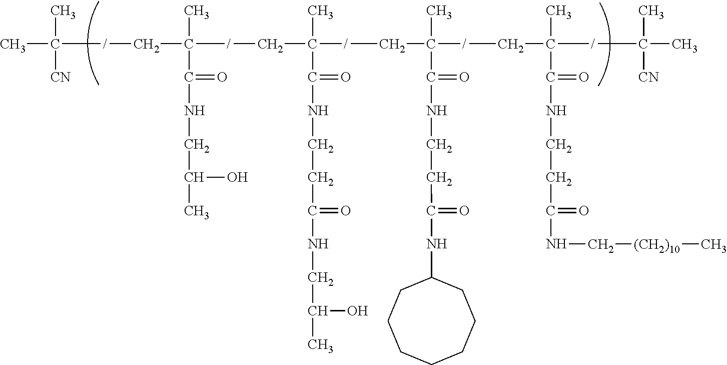

The polymer precursor poly(HPMA-co-Ma-β-Ala-TT) (0.2 g, Mw=37,700 g/mol, Ð=2.04, 12.84% mol TT), prepared according to Example 3, was dissolved in 0.7 mL of DMSO and 93 uL (4.4 mg) of stock dodecyl-amine solution (23.66 mg/500 uL CHCl₃) and 125 uL (2.18 mg) of stock aminocyclooctane solution (8.7 mg/500 uL DMSO) were added to the solution. N,N-diisopropylethylamine (DIPEA) (14.9 μL) was then added and the reaction mixture was stirred for 4 h at laboratory temperature. Then 1-amino-propan-2-ol (10 μL) was added to the solution and the reaction mixture was stirred for 10 min. Then, the polymer conjugate 11, poly(HPMA-co-Ma-3-Ala-dodecyl-amino-co-Ma-β-Ala-aminocyclooctane), was isolated by precipitation into a mixture of acetone:diethyl ether (3:1), filtered off, washed with acetone and diethyl ether, and dried under vacuum. The content of the hydrophobic co-monomer unit (containing dodecylamine) 0.7 mol % and the content of the co-monomer unit containing aminocycloacetate 0.85 mol % were determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 11.29 mol %. The molecular weight of the conjugate Mw 43,000 and dispersity 1.43 were determined using a Shimadzu HPLC equipped with multi-angle scattering, viscometer and differential refractive index detectors in PBS buffer.

### Example 17

### Preparation of Poly(HPMA-co-Ma-β-Ala-dodecyl-amino-co-Ma-β-Ala-norbornen-2-methylamine) Polymer Conjugate (Conjugate 12)

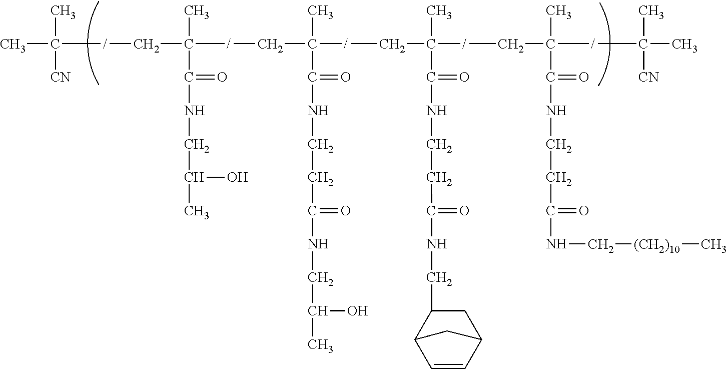

The polymer precursor poly(HPMA-co-Ma-β-Ala-TT) (0.2 g, Mw=37,700 g/mol, Ð=2.04, 12.84% mol TT), prepared according to Example 3, was dissolved in 1.0 mL of DMSO and 93 uL (4.4 mg) of stock dodecylamine solution (23.66 mg/500 uL CHCl₃) and 84 uL (2.1 mg) of stock 5-norbornene-2-methylamine solution (4.99 mg/200 uL DMSO) were added to the solution. N,N-diisopropylethylamine (DIPEA) (14.8 μL) was then added and the reaction mixture was stirred for 4 h at laboratory temperature. Then 1-amino-propan-2-ol (10 μl) was added to the solution and the reaction mixture was stirred for 10 min. Then the polymer conjugate 12, poly (HPMA-co-Ma-β-Ala-dodecyl-amine-co-Ma-β-Ala-5-norbornene-2-methylamine), was isolated by precipitation into a mixture of acetone:diethyl ether (3:1), filtered off, washed with acetone and diethyl ether and dried under vacuum. The content of the hydrophobic co-monomer unit (containing dodecylamine) 0.75 mol % and the content of the co-monomer unit containing 5-norbornene-2-methylamine 2.5 mol % were determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 9.59 mol %. The molecular weight of the conjugate Mw 43,000 and dispersity 1.45 were determined using a Shimadzu HPLC equipped with multi-angle scattering, viscometer and differential refractive index detectors in PBS buffer.

### Example 18

### Preparation of Poly(HPMA-co-Ma-β-Ala-dodecyl-amino-co-Ma-β-Ala-(2,6-N-(2-aminoethyl)octahydroxyclopentapyrrole-2-karboxamide) Polymer Conjugate (Conjugate 13)

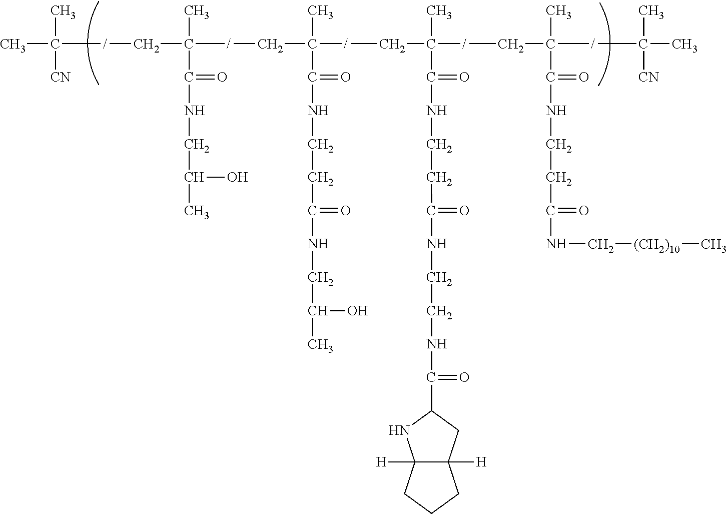

The polymer precursor poly(HPMA-co-Ma-β-Ala-TT) (0.2 g, Mw=37,700 g/mol, Ð=2.04, 12.84% mol TT), prepared according to Example 3, was dissolved in 1.0 mL of DMSO and 93 uL (4.4 mg) of stock dodecyl-amine solution (23.66 mg/500 uL CHCl₃) and 3.4 mg of (2,6)-N-(2-aminoethyl)octahydrocyclopentapyrrole-2-carboxamide was added. N,N-diisopropylethyl-amine (DIPEA) (15.3 μL) was then added and the reaction mixture was stirred for 4 h at laboratory temperature. Then 1-amino-propan-2-ol (10 μL) was added to the solution and the reaction mixture was stirred for 10 min. Then, the polymer conjugate 13, poly(HPMA-co-Ma-β-Ala-dodecyl-amino-co-Ma-b-Ala-(2,6-N-(2-aminoethyl)octahydrocyclopentapyrrole-2-carboxamide), was isolated by precipitation into a mixture of acetone:diethyl ether (3:1), filtered off, washed with acetone and diethyl ether, and dried under vacuum. The content of the hydrophobic co-monomer unit (containing dodecylamine) 0.75 mol % and the content of the co-monomer unit containing N-(2-aminoethyl)octahydrocyclopentapyrrole-2-carboxamide 1.8 mol % were determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (III) units was 10.29 mol %. The molecular weight of the conjugate Mw 43,000 and dispersity 1.45 were determined using a Shimadzu HPLC equipped with multi-angle scattering, viscometer and differential refractive index detectors in PBS buffer.

### Example 19

### Preparation of dodecylamine-poly(HPMA)-biotin Homopolymers by RAFT Polymerisation—Conjugates 14 and 15

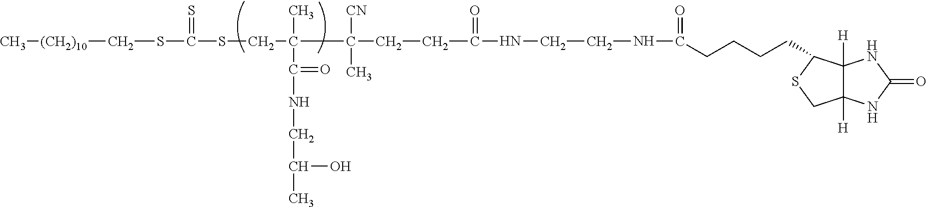

Polymer dodecylamine-poly(HPMA)-biotin conjugates were prepared by reversible addition-fragmentation chain-transfer (RAFT) polymerisation. Preparation of Conjugate 14: 0.4 g of HPMA was dissolved in 3.4 mL of tert-butanol and 18.8 mg of N-[2-[5-[(3aR,4R,6aS)—2-oxo-1,3,3a,4,6,6a-hexahydrothieno[3,4-d]imidazol-4-yl]pentanoylamino]ethyl]-4-cyano-4-dodecyl-sulfanylcarbothioylsulfanyl-pentanamide prepared in Example 13, dissolved in 425 uL of DMAc, and 4.31 mg of 2,2′-azobis(4-methoxy-2,4-dimethylvaleronitrile) were added to the solution and the solution was transferred to a polymerisation ampoule. The mixture was bubbled for 10 min with argon and then the ampoule was sealed. The polymerisation reaction was carried out at 30° C. (72 h). The polymer precursor was isolated by precipitation into a mixture of acetone:diethyl ether (3:1), filtered off, washed with acetone and diethyl ether and dried under vacuum. This procedure afforded a polymer dodecylamine-poly(HPMA)-biotin conjugate with molecular weight Mw=22,400 g/mol, polydispersity D=1.18 and containing one dodecylamine molecule at the alpha terminus of the polymer chain and one biotin molecule at the omega terminus of the polymer chain. Similarly, Conjugate 15 was prepared having Mw=42,000 g/mol, polydispersity D=1.17.

### Example 20

### Preparation of dodecylamine-poly(HPMA) Homopolymer by RAFT Polymerisation—Conjugate 16

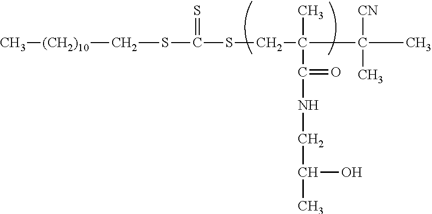

The polymer dodecylamine-poly(HPMA) conjugate was prepared by reversible addition-fragmentation chain-transfer (RAFT) polymerisation. 0.4 g of HPMA was dissolved in 3.4 mL of tert-butanol and 3.9 mg of 2-cyanoprop-2-yl)-dodecyltrithiocarbonate dissolved in 64 μL of DMAc and 1.72 mg of 2,2′-azobis(4-methoxy-2,4-dimethylvaleronitrile) were added to the solution and the solution was transferred to a polymerisation ampoule. The mixture was bubbled for 10 min with argon and then the ampoule was sealed. The polymerisation reaction was carried out at 40° C. (24 h). The polymer precursor was isolated by precipitation into a mixture of acetone:diethyl ether (3:1), filtered off, washed with acetone and diethyl ether and dried under vacuum. This procedure afforded a polymer dodecylamine-poly(HPMA) conjugate with molecular weight Mw=42,400 g/mol, polydispersity D=1.12 and containing one dodecylamine molecule at the alpha terminus of the polymer chain.

### Example 21

### Preparation of Copolymer Based on 2-oxazoline with poly(2-methyl-oxazolin-co-N-ethyl-4-oxo-4-(2-thioxothiazolidin-3-yl)butanamide) Structure

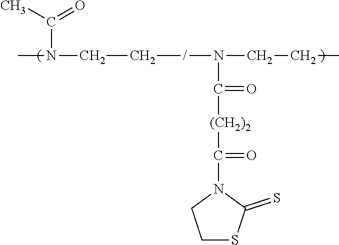

In a flask, under argon atmosphere, 2-methyl-2-oxazoline (8.6 g, 101 mmol), methyl 3-(4,5-dihydrooxazol-2-yl)propanoate (1.73 g, 11 mmol) and 2-phenyl-2-oxazoliniumtetrafluoroborate initiator (39.6 mg, 168 mol, [M]/[I]=600) were dissolved in 25.1 mL of freshly distilled sulfolane to give a monomer concentration of 3M. The reaction mixture was stirred at 60° C. in a glove box for 2 weeks, and then terminated overnight with solid sodium azide (33 mg, 504 mol). The polymer mixture was diluted with distilled water (10 mL) and dialysed against the same solvent (MWCO 3 kDa). Subsequently, the polymer was isolated by lyophilisation. Hydrolysis of the methyl ester group was carried out in a water:methanol (1:1) mixture at pH 12 using 0.1 M NaOH. After hydrolysis, the methanol was removed by evaporation and the aqueous solution was acidified with 0.1 M HCl to pH 2. The polymer was diluted with distilled water (10 mL) and dialysed against the same solvent (MWCO 3 kDa) and then lyophilised. In the next step, 1 g of polymer was dissolved together with 0.15 g of thiazolidine-2-thine in 10 mL of DMSO and 0.3 g of EDC·HCl was added to the solution. The reaction mixture was stirred for 24 h. Poly(2-methyl-oxazolin-co-N-ethyl-4-oxo-4-(2-thioxothiazolidin-3-yl)butanamide) was precipitated into a 2:1 acetone:diethyl ether mixture, filtered off, washed with acetone and diethyl ether and dried under vacuum. The resulting polymer was obtained as a yellowish powder (yield 71 wt %).

The molecular weight of the conjugate Mw 12,400 and dispersity 1.13 were determined using a Shimadzu HPLC equipped with multi-angle scattering, viscometer and differential refractive index detectors in PBS buffer. The content of reactive thiazolidine-2-thione (TT) groups was 9.8 mol %.

### Example 22

### Preparation of poly(2-methyl-oxazolin-co-N′-dodecyl-N-ethyl-butanediamide-co-N′-2-hydroxypropyl-N-ethyl-butanediamide) Conjugate—Conjugate 17

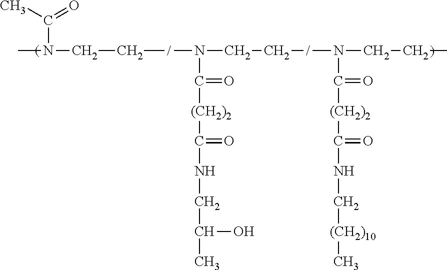

The polymer precursor poly(2-methyl-oxazolin-co-N-ethyl-4-oxo-4-(2-thioxothiazolidin-3-yl)butanamide) (0.2 g, Mw=12,400 g/mol, Ð=1.13, 9.8% mol TT), prepared according to Example 21, was dissolved in 1.0 mL of DMSO and 166.3 uL (3.0 mg) of stock solution of dodecylamine in CHCl₃ (9.02 mg/500 uL CHCl₃) was added to the solution. N,N-diisopropylethylamine (DIPEA) (7.05 μL) was then added and the reaction mixture was stirred for 4 h at laboratory temperature. Then 1-amino-propan-2-ol (10 μL) was added to the solution and the reaction mixture was stirred for 10 min. Poly(2-methyl-oxazoline-co-N′-dodecyl-N-ethyl-butanediamide-co-N′-2-hydroxypropyl-N-ethyl-butanediamide) was isolated by precipitation into a mixture of acetone:diethyl ether (3:1), filtered off, washed with acetone and diethyl ether and dried under vacuum. The content of the hydrophobic co-monomer unit (containing 0.75 mol % dodecylamine) was determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (IV) units was 9.05 mol %. The molecular weight of the conjugate Mw 14,200 and dispersity 1.15 were determined using a Shimadzu HPLC equipped with multi-angle scattering, viscometer and differential refractive index detectors in PBS buffer.

### Example 23

### Preparation of Poly(2-methyl-oxazolin-co-N′-dodecyl-N-ethyl-butanediamide-co-N′-cyclooctyl-N-ethyl-butanediamide-co-N′-2-hydroxypropyl-N-ethyl-butanediamide) Polymer Conjugate—Conjugate 18

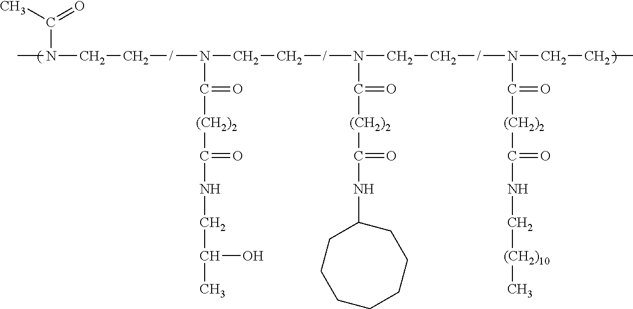

The polymer precursor poly(2-methyl-oxazolin-co-N-ethyl-4-oxo-4-(2-thioxothiazolidin-3-yl)butanamide) (0.2 g, Mw=12,400 g/mol, Ð=1.13, 9.8% mol TT), prepared according to Example 21, was dissolved in 1.0 mL of DMSO and 166.3 uL (3.0 mg) of stock solution of dodecylamine in CHCl₃ (9.02 mg/500 uL CHCl₃) and 84 uL (2.1 mg) of stock solution of cyclooctylamine in DMSO (4.99 mg/200 uL DMSO) were added to the solution. N,N-diisopropylethylamine (DIPEA) (15.4 μL) was then added and the reaction mixture was stirred for 4 h at laboratory temperature. Then 1-amino-propan-2-ol (10 μL) was added to the solution and the reaction mixture was stirred for 10 min. Then, the polymer conjugate poly(2-methyl-oxazoline-co-N′-dodecyl-N-ethyl-butanediamide-co-N′-cyclooctyl-N-ethyl-butanediamide-co-N′-2-hydroxypropyl-N-ethyl-butanediamide) was isolated by precipitation into a mixture of acetone:diethyl ether (3:1), filtered off, washed with acetone and diethyl ether, and dried under vacuum. The content of hydrophobic co-monomer unit (containing dodecylamine) of 0.75 mol % and the cyclooctylamine-containing co-monomer unit content of 2.2 mol % were determined in the hydrolysate of the sample (6N HCl, 115° C., 16 h) by HPLC with fluorescence detector (Ex. 229 nm, Em. 490 nm) on a Chromolith C18 column by pre-column derivatisation with o-naphthalenedialdehyde. The content of type (IV) units was 6.85 mol %. The molecular weight of the conjugate Mw 15,300 and dispersity 1.18 were determined using a Shimadzu HPLC equipped with multi-angle scattering, viscometer and differential refractive index detectors in PBS buffer.

### Example 24

### Preparation of 2-methyl-N-tetradecyl-prop-2-enamide (Ma-tetradecylamine)

Tetradecylamine (4.0 g, 18.8 mmol) was dissolved in 40 mL ethyl acetate and the inhibitor 2.5-di-tert-butylhydroquinone was added to the solution. Methacryloyl anhydride (2.9 g, 18.8 mmol) was diluted with 3 mL of ethyl acetate and slowly added dropwise to the tetradecylamine solution at laboratory temperature so as not to exceed 25° C. After addition of all anhydride, the reaction mixture was stirred for 1 h at laboratory temperature. The reaction mixture was shaken 3times with 50 mL of 2% NaHCO₃ and 50 mL of H₂O. The reaction mixture was dried with anhydrous Na₂SO₄ and the product was crystallised in a freezer. The precipitated crystals were filtered off, washed with chilled ethyl acetate and dried under vacuum. 3.5 g (66.1%) of 2-methyl-N-tetradecyl-prop-2-enamide monomer was obtained. Characterisation by HPLC on a Chromolith High Resolution RP18e column showed a single peak with a retention time of 13.8 min at 220 nm. CHN analysis C=76.81 (76.90 found); H=12.53 (12.54 found); N=4.98 (5.04 found).

### Example 25

### Preparation of Poly(HPMA-co-Ma-tetradecylamine) conjugate by radical precipitation Copolymerisation

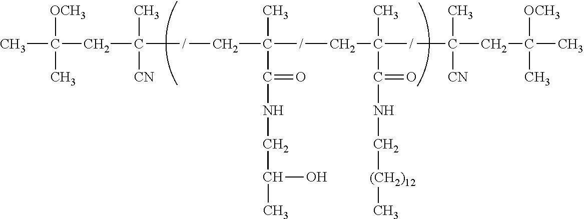

The poly(HPMA-co-Ma-tetradecylamine) conjugate was prepared by radical precipitation copolymerisation of HPMA with 2-methyl-N-tetradecyl-prop-2-enamide, according to Example 24, in acetone at 40° C. for 24 h. The total concentration of co-monomers in the polymer mixture was 12.5 wt % and the concentration of the initiator 2,2′-azobis[N-(2-carboxyethyl)-2-methylpropionamidine (V-70) was 0.5 wt %. HPMA (0.3 g, 2.1 mmol), 2-methyl-N-tetradecyl-prop-2-enamide (0.025 g, 0.087 mmol) and AIBN (13 mg, 0.04 mmol) were dissolved in 2.9 mL of acetone. The solution was bubbled with argon for 10 min. Polymerisation was carried out in a sealed ampoule at 40° C. for 24 h. The precipitated copolymer was filtered off, washed with acetone and diethyl ether and dried under vacuum. 0.196 g of copolymer (60.4%) with a molecular weight of Mw 44,300 and a dispersity of 2.21 was obtained. The content of the hydrophobic co-monomer unit (derived from 2-methyl-N-tetradecyl-prop-2-enamide) was 1.0 mol %.

### Example 26

### Suppression of Non-Specific Sorption of IgG-HRP Conjugate to the Surface of ELISA Plate Wells

A concentrate of a polyclonal rabbit antibody conjugate with horseradish peroxidase prepared by the glutaraldehyde method was diluted 500-fold in phosphate buffer containing various amounts of BSA, biotinylated BSA, Conjugate 1 from Example 4 and Conjugate 3 from Example 6. Each test solution was dosed into a 96-well polystyrene microtiter plate (Greiner), four wells at 200 uL per well. The plate was then left for 60 minutes on the bench, at laboratory temperature and without shaking. After aspirating and re-washing the wells (3×350 uL) with a buffer containing Tween-20 and NaCl, a colorimetric reaction was induced by adding 200 uL of TMB. After 10 minutes, 20 the colorimetric reaction was stopped by the addition of 50 uL of 2 M HCl.

The absorbance (optical density—OD) was then measured at a wavelength of 450 nm. The results are shown in Table 1.

TABLE 1
Results of suppression of non-specific sorption by commercial BSAs and Conjugates 1 and 3
Concentration of tested blockers in the phosphate buffer, g/L

| | BSA (g/L) | | | biot. BSA (g/L) | | | Conjugate 1 (g/L) | | | Conjugate 3 (g/L) | | |
|---|---|---|---|---|---|---|---|---|---|---|---|---|
| | 0.2 | 1 | 5 | 0.1 | 0.5 | 2 | 0.2 | 1 | 5 | 0.1 | 0.5 | 2 |
| | OD₄₅₀ | | | | | | | | | | | |
| | 3.190 | 0.654 | 0.077 | 2517 | 0.540 | 0.089 | 0.394 | 0.105 | 0.049 | 1.002 | 0.165 | 0.087 |
| | 2.760 | 0.609 | 0.068 | 2.435 | 0.490 | 0.098 | 0.376 | 0.128 | 0.047 | 0.997 | 0.194 | 0.088 |
| | 3.048 | 0.691 | 0.069 | 2.488 | 0.504 | 0.105 | 0.390 | 0.129 | 0.046 | 0.976 | 0.184 | 0.105 |
| | 2.882 | 0.782 | 0.075 | 2.564 | 0.514 | 0.130 | 0.383 | 0.137 | 0.047 | 0.947 | 0.169 | 0.104 |
| Mean | 2.970 | 0.684 | 0.072 | 2.501 | 0.512 | 0.105 | 0.385 | 0.125 | 0.047 | 0.981 | 0.178 | 0.096 |
| C.V., % | 6.34 | 10.75 | 6.13 | 2.15 | 4.05 | 16.87 | 2.11 | 1090 | 2.37 | 2.56 | 7.62 | 10.18 |

OD values at concentrations of BSA, 1.0 g/L, and Conjugate 1, 0.2 g/L, indicate that Conjugate 1 reduces the non-specific sorption of the used IgG-HRP conjugate at least 5 times more than BSA. In the case of biotinylated BSA and Conjugate 3 also containing biotin, a comparison of the OD at concentrations of 0.1 and 0.5 g/L of the two blockers shows that the OD values for Conjugate 3 are at least 2.5 times smaller. It is also apparent that the introduction of biotin will improve the blocking effect of BSA approximately 2×.

### Example 27

### Model ELISA System for the Determination of TSH in Blood Serum

A sandwich ELISA system for the determination of human Thyroid Stimulating Hormone (TSH) was constructed from two commercially available mouse monoclonal antibodies. One monoclonal antibody was conjugated with biotin and the other with horseradish peroxidase.

Preparation of the Conjugate with Biotin:

1 mg of antibody in 1 mL of borate buffer pH 8.5 is biotinylated with 4% (w/w) Biotin-NHS, which is added at a concentration of 10 mg/mL in DMF. The biotinylation takes place for 20 min and is stopped by the addition of 100 uL of 0.2 M NH₄Cl solution. Subsequent dialysis separates the free biotin.

Preparation of the Conjugate with Horseradish Peroxidase:

1 mg of antibody is conjugated with 5 mg of horseradish peroxidase (HRP) using glutaraldehyde. First, HRP is activated with a 1.25% glutaraldehyde solution in phosphate buffer pH 6.8 for 14 hours. After gel chromatography (Sephadex 25) to remove free glutaraldehyde, the activated HRP is mixed with a solution of IgG in 0.15 m NaCl with the addition of 5% (v/v) carbonate-bicarbonate buffer pH 9.5. The reaction is stopped after 14 hours by the addition of a 5% (v/v) solution of 0.2M lysine. The IgG-RP conjugate is separated by gel chromatography on a Superdex HR 200 cartridge column.

The conjugates were then used to construct two sandwich ELISA systems.

System with BSA

A biotin-labelled monoclonal antibody is attached to the walls of a microtiter plate using biotinylated BSA:

First, 300 uL of biotinylated BSA solution is pipetted into the wells of a microtiter plate (Greiner) at a concentration of 10 μg/mL in phosphate buffer. The solution is left in the wells at laboratory temperature until the next day. The contents of the wells are thoroughly removed by suction and 300 μL of streptavidin solution with a concentration of 1 μg/mL in phosphate buffer is pipetted. Incubation takes place again at laboratory temperature until the next day, then the solution is thoroughly removed by suction and 250 μL of a biotinylated monoclonal antibody solution with a concentration of 1 μg/mL in a phosphate buffer containing also BSA at a concentration of 1 mg/mL is pipetted. This solution is left in the wells for 48 hours, followed by thorough suction and drying for 4 hours. The plates are thus ready for use and can be stored in the refrigerator for 6 months. The IgG-HRP conjugate is diluted 20,000× in citrate buffer containing BSA at a concentration of 1 mg/mL.

System with Conjugates 2 and 3

In this case, a biotin-labelled monoclonal antibody is attached to the wells using Conjugates 2 and 3. The procedure for coating the wells is identical to the BSA system, but with two changes: (i) instead of 10 μg/mL biotinylated BSA, Conjugate 3 is used at a concentration of 0.1 μg/mL; (ii) the biotinylated antibody coating solution contains Conjugate 2 instead of BSA, also at a concentration of 1 mg/mL.

The IgG-RP conjugate is also diluted 20,000× in citrate buffer, which however contains Conjugate 2 at a concentration of 1 mg/mL.

The same calibrators were used in both systems, i.e. solutions of human TSH (Sigma-Aldrich no. T9265) in bovine serum; their concentrations were determined using the WHO reference preparer (NIBSC 80/558). The working protocol was also the same in both systems:
50 uL of calibrator or sample+150 uL of IgG-HRP conjugate solution is pipetted into the well
120 min incubation at laboratory temperature, without shaking
Removal by suction, wash 3times with washing solution
200 uL TMB, 10 min at laboratory without access to light
50 μL of 2 M HCl
Measurement of optical density at 450 nm (OD₄₅₀)

The following Table 2 shows the OD₄₅₀ values of the calibrators for both systems, each calibrator was measured in two wells in each system.

TABLE 2
Calibration dependence of the TSK calibrator when using blockers
based on commercial BSA and the combination of Conjugate 2 and 3

| TSH calibrator, | System with BSA | | System with Conjugates 2 and 3 | |
|---|---|---|---|---|
| mIU/L | Mean OD₄₅₀ | C.V., % | Mean OD₄₅₀ | C.V., % |
| 0 | 0.043 | 2.64 | 0.041 | 0.17 |
| 0.1 | 0.048 | 1.18 | 0.051 | 1.96 |
| 0.5 | 0.082 | 0.52 | 0.093 | 1.06 |
| 2.5 | 0.269 | 2.50 | 0.330 | 3.92 |
| 10 | 0.899 | 3.30 | 1.123 | 1.53 |
| 50 | 3.416 | 1.51 | 3.827 | 1.83 |

The OD values of both calibration dependences are comparable, for the system with Conjugates 2 and 3 the calibration responses for the calibrators 2.5, 10 and 50 mIU/L are even demonstrably higher, as well as the values of C.V. duplicates of individual calibrators are comparable. It is important to note that to achieve the same calibration curve, it is necessary to use a 100-fold lower concentration of conjugate 3 than biotinylated BSA.

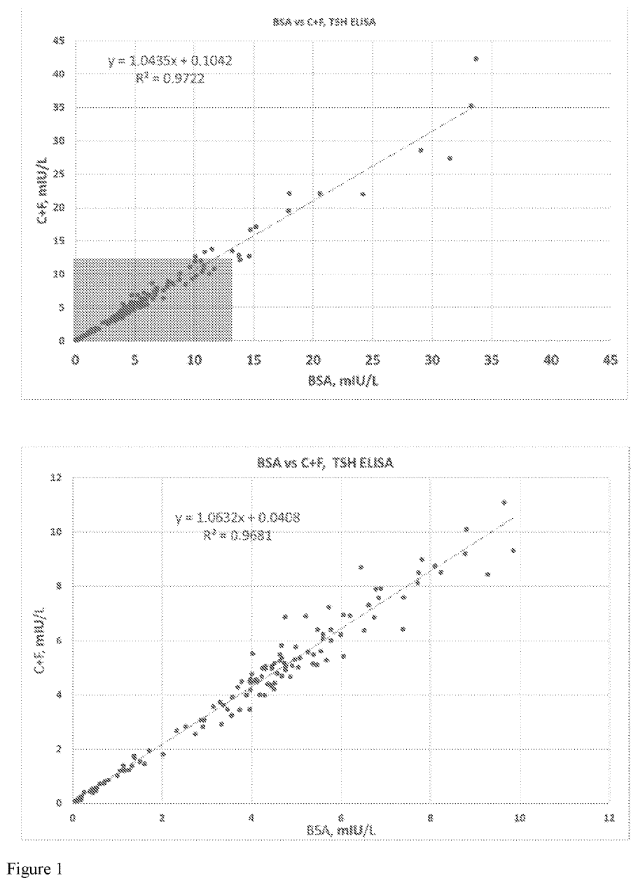

In both systems, 500 real samples obtained from an endocrinology clinic were analysed, the obtained results are compared graphically in FIGS. 1A and 1B. It is clear that the use of the system with Conjugates 2 and 3 allows achieving the same results on real samples as the BSA system. Here, too, it is necessary to realise that Conjugate 3 is used at a concentration 100 times lower than that of BSA.

### Example 28

### Suppression of Non-Specific Sorption of HRP to the Surface of ELISA Plate Wells

A solution of horseradish peroxidase with a concentration of 5 mg/L in phosphate buffer containing BSA, Conjugate 2 (Example 5) and Conjugate 11 (Example 16) with a concentration of 0.1 g/L was dosed into a 96-well polystyrene microtiter plate (Greiner). Each solution was dosed into eight wells in a volume of 200 μL per well. The plate was then left for 16 hours on the workbench, at laboratory temperature and without shaking. After removing with suction and repeatedly washing the wells (3×350 μL) with buffer containing Tween 20 and NaCl, the colorimetric reaction was induced by adding 200 μL TMB. After 10 minutes, the colorimetric reaction was stopped by the addition of 50 μL of 2M HCl. Subsequently, the absorbance was measured at a wavelength of 450 nm. The results are shown in Table 3.

TABLE 3
Absorbance values of HHP non-specific sorption suppression
experiment with BSA, Conjugate 2 and Conjugate 11

| | | |
|---|---|---|
| BSA | Conjugate 2 | Conjugate 11 |

OD₄₅₀

| | | |
|---|---|---|
| Over, | 0.405 | 0.220 |
| OD ≥ 3.6 | 0.356 | 0.210 |
| | 0.358 | 0.232 |
| | 0.300 | 0.195 |
| | 0.365 | 0.206 |
| | 0.393 | 0.200 |
| | 0.363 | 0.311 |
| | 0.434 | 0.253 |
| Mean | 0.372 | 0.228 |
| C.V. % | 10.74 | 16.77 |

It is evident from the OD values that the blocking effect for the sorption of horseradish peroxidase increases in the order of BSA-Conjugate 2-Conjugate 11. While BSA at a concentration of 0.1 mg/mL was unable to prevent HRP from non-specifically binding to the surface of the plate, Conjugates 2 and 11 at this small concentration already effectively limited the non-specific sorption. Conjugate 11 containing, in addition to a hydrophobically active anchor, an interaction-limiting ligand, demonstrated an increased ability to block the non-specific interaction of HRP on the plate surface.

### Example 29

### Competitive Sorption of Conjugates 14 and 15 Versus BSA and Biotinylated BSA Versus Conjugate 16 to the Surface of the ELISA Plate Wells

A phosphate buffer with a conjugate containing biotin (Conjugates 14 and 15 from Example 19) was dosed into a 96-well polystyrene microtiter plate (Greiner), the concentration of the conjugate was 1 mg/L, BSA of different concentration was used as a blocker. Alternatively, the phosphate buffer contained biotinylated BSA at a concentration of 1 mg/mL and a biotin-free conjugate (Conjugate 16 from Example 20) was used as a blocker. Each solution was dosed into eight wells in a volume of 200 μL per well. The plate was then left for 16 hours on the workbench, at laboratory temperature and without shaking. Subsequently, the solution was thoroughly removed by suction, 200 μL of phosphate buffer containing 0.1% fish gelatine was dosed into all wells, and the incubation took place again overnight. After suction and repeated washing of the wells (3×350 μL) with a buffer containing Tween 20 and NaCl, a 200 μL solution of commercial streptavidin conjugate with horseradish peroxidase (STR-HRP) diluted in phosphate buffer with 0.10 fish gelatine was dosed into the wells. After one hour of incubation at laboratory temperature and without shaking, suction and repeated washing (3×350 μL) were performed and the colorimetric reaction was induced by adding 200 μL of TMB. After 10 minutes, the colorimetric reaction was stopped by the addition of 50 μL of 2M HCL. Subsequently, the absorbance was measured at a wavelength of 450 nm, the results are shown in Table 4.

TABLE 4
Results of sorption properties of Conjugates 14 to 16

| | Sorption of Conjugate 14 | | | Sorption of Conjugate 15 | | | Sorption of biotinylated | | |
|---|---|---|---|---|---|---|---|---|---|
| | at a concentration of 1 | | | at a concentration of 1 | | | BSA at a concentration | | |
| | mg/L from a PBS | | | mg/L from a PBS | | | of 1 mg/L from a PBS | | |
| | solution under different | | | solution under different | | | solution under different | | |
| | concentrations of the | | | concentrations of the | | | concentrations of | | |
| | BSA blocker | | | BSA blocker | | | Conjugate 16 blocker | | |
| | Blocker concentration, mg/L | | | | | | | | |
| | 0 | 2 | 50 | 0 | 2 | 50 | 0 | 0.5 | 1 |
| | OD₄₅₀ | | | | | | | | |
| | 3.751 | 3.733 | 1.579 | 3.477 | 3.390 | 1.735 | 2.921 | 1.985 | 1.341 |
| | 3.956 | 3.559 | 1.545 | 3.693 | 3.530 | 1.807 | 2.880 | 1.799 | 1.235 |
| | 3.952 | 3.118 | 1.686 | 3.776 | 3.475 | 1.874 | 2.977 | 1.721 | 1.142 |
| | 3.691 | 3.390 | 1.559 | 3.691 | 2.969 | 1.735 | 2.800 | 1.599 | 1.061 |
| | 3.931 | 3.625 | 1.711 | 3.660 | 3.234 | 1.815 | 2.801 | 1.577 | 1.135 |
| | 3.839 | 3.538 | 1.616 | 3.596 | 3.380 | 1.772 | 2.810 | 1.694 | 1.240 |
| | 3.839 | 3.742 | 1.742 | 3.566 | 3.400 | 1.809 | 2.766 | 1.642 | 1.235 |
| | 3.962 | 3.786 | 1.654 | 3.740 | 3.536 | 1.745 | 2.779 | 1.772 | 1.235 |
| Mean | 3.865 | 3.561 | 1.637 | 3.650 | 3.364 | 1.787 | 2.841 | 1.724 | 1.203 |
| C.V., % | 2.67 | 6.22 | 4.48 | 2.69 | 5.56 | 2.73 | 2.66 | 7.62 | 7.15 |
| Mean | 1.00 | 0.92 | 0.42 | 1.00 | 0.92 | 0.49 | 1.00 | 0.61 | 0.42 |

In the mentioned test, the measured signal is the result of binding the STR-HRP conjugate to biotin attached to the solid phase during the incubation of the first sorption buffer, i.e. solutions of Conjugates 14 and 15, or biotinylated BSA. It is clear from the results that in order for the sorption of Conjugates 14 and 15 to decrease by more than 50%, the concentration of the competitive blocker, i.e. BSA, needs to be 50 mg/L, in the case of Conjugate 16 as a biotinylated BSA sorption blocker, a concentration of 1 mg/mL is sufficient. These results uniformly demonstrate significantly better sorption properties of the tested conjugates compared to BSA.

### Example 30

### Suppression of Non-Specific Sorption of IgG-HRP Conjugate to the Surface of Paramagnetic Microparticles

The conjugate concentrate of polyclonal rabbit antibody with horseradish peroxidase prepared by the glutaraldehyde method was diluted 500× in phosphate buffer with BSA, Conjugate 2 (Example 5), Conjugate 11 (Example 16) and Conjugate 12 (Example 17), the concentration of the blocker was always 0.1 g/L. Invitrogen paramagnetic particle (PMP) suspension no. 11201D with a volume of 5 μL was mixed with 600 μL of washing phosphate solution in a small container with a conical bottom, a strong magnet was attached to the wall of the container to separate the PMP, after the separation of the PMP the liquid volume was removed by suction, this washing step was repeated. Subsequently, 300 uL of the conjugate solution with the tested blocker was added, the suspension was left for 15 minutes at laboratory temperature. After magnetic separation of the PMPs and suction of the solution, the PMPs were washed twice in the manner already described. In the last step, colorimetric substrate TMB in a volume of 200 μL was added to PMPs. After 10 minutes, 150 μL of the solution was transferred to a clean well of a 96-well microtiter plate, and STOP solution in a volume of 50 μL was immediately added. The absorbance (optical density—OD) was measured at a wavelength of 450 nm. The entire process was repeated three times for each blocker tested. The results are shown in Table 5.

TABLE 5
Results of blocking the non-specific sorption
of IgG-HRP to the PMP surface

| | Blocker at a concentration of 0.1 g/L | | | |
|---|---|---|---|---|
| | BSA | Conjugate 2 | Conjugate 11 | Conjugate 12 |
| | OD₄₅₀ | | | |
| | 2.893 | 0.469 | 0.878 | 1.051 |
| | 3.030 | 0.537 | 1.090 | 1.007 |
| | 3.944 | 0.599 | 1.087 | 0.931 |
| Mean | 3.289 | 0.535 | 1.018 | 0.996 |
| C.V., % | 14.19 | 9.92 | 9.75 | 4.94 |

The blocking effect of the non-specific sorption of the IgG-HRP conjugate was determined for the BSA control sample and three selected blockers. From the results shown in Table 5, it is clear that all tested synthetic blockers show several times better blocking activity than BSA alone. It has thus been shown that synthetic blockers have a significantly better blocking effect than the commonly used BSA.

### Example 31

### Suppression of Non-Specific Sorption of IgG-HRP Conjugate to the Surface of ELISA Plate Wells—Comparison of Multiple Conjugates

The conjugate concentrate of polyclonal rabbit antibody with horseradish peroxidase, prepared by the glutaraldehyde method, was diluted 500 times in phosphate buffer with a BSA content of 1 g/L, and eleven selected conjugates with a content 10 times smaller, i.e. 0.1 g/L. Each tested solution was dosed into a 96-well polystyrene microtiter plate (Greiner), each time into eight wells in a volume of 200 uL per well. The plate was then left for 60 minutes on the workbench, at laboratory temperature and without shaking. After removal by suction and repeatedly washing the wells (3×350 μL) with buffer containing Tween 20 and NaCl, the colorimetric reaction was induced by adding 200 μL TMB. After 10 minutes, the colorimetric reaction was stopped by the addition of 50 μL of 2M HCl. Subsequently, the absorbance (optical density—OD) was measured at a wavelength of 450 nm. The results are shown in Table 6.

TABLE 6
Results of the blocking of non-specific sorption of IgG-HRP to the
surface of the wells of the ELISA plate - comparison of conjugates
(only conjugate numbers are given in the description of the table)

| | BSA | 5 | 18 | 4 | 2 | 9 | 17 | 1 | 11 | 12 | 13 | 14 |
|---|---|---|---|---|---|---|---|---|---|---|---|---|
| | 1 g/L | 0.1 g/L | | | | | | | | | | |
| | OD₄₅₀ | | | | | | | | | | | |
| | 0.699 | 0.992 | 0.987 | 0.850 | 0.687 | 0.593 | 0.288 | 0.538 | 0.592 | 0.805 | 0.895 | 0.521 |
| | 0.555 | 0.967 | 1.118 | 0.881 | 0.664 | 0.586 | 0.254 | 0.515 | 0.652 | 0.800 | 0.851 | 0.540 |
| | 0.879 | 1.051 | 1.138 | 1.026 | 0.698 | 0.578 | 0.251 | 0.510 | 0.565 | 0.789 | 0.811 | 0.527 |
| | 1.012 | 1.034 | 1.025 | 0.938 | 0.699 | 0.662 | 0.264 | 0.522 | 0.592 | 0.665 | 0.738 | 0.536 |
| | 0.995 | 1.003 | 1.016 | 0.920 | 0.682 | 0.682 | 0.242 | 0.516 | 0.688 | 0.640 | 0.712 | 0.512 |
| | 1.004 | 1.003 | 1.140 | 0.970 | 0.720 | 0.710 | 0.242 | 0.527 | 0.550 | 0.717 | 0.770 | 0.527 |
| | 0.737 | 0.990 | 1.143 | 0.963 | 0.651 | 0.672 | 0.244 | 0.519 | 0.611 | 0.665 | 0.698 | 0.517 |
| | 0.862 | 0.984 | 1.093 | 0.967 | 0.680 | 0.604 | 0.248 | 0.503 | 0.543 | 0.626 | 0.732 | 0.519 |
| Mean | 0.843 | 1.003 | 1.083 | 0.939 | 0.685 | 0.636 | 0.254 | 0.519 | 0.599 | 0.714 | 0.776 | 0.525 |
| C.V., % | 19.77 | 2.73 | 5.87 | 5.90 | 3.12 | 8.01 | 6.18 | 2.08 | 8.40 | 10.51 | 9.05 | 1.79 |

All tested conjugates achieved a comparable or even significantly better blocking effect at a concentration 10times lower than that of BSA. It is also worth mentioning the demonstrably better reproducibility of the measured signal of the adsorbed IgG-HRP conjugate for the tested synthetic blockers compared to BSA.

### Example 32

### Preparation of N-[3-(cyclooctylamino)-3-oxo-propyl]-2-methyl-prop-2-enamide Monomer

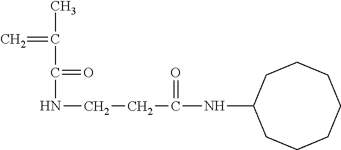 [SMILES: C=C(C)C(=O)NCCC(=O)NC1CCCCCCC1]

3-Methacrylamidopropanoic acid (Ma-β-Ala-OH, 2 g) and NH₂-cyclooctylamine (1.62 g) were dissolved in 10 mL of dichloromethane (DCM) and a catalytic amount of 4-dimethylaminopyridine (DMAP) was added to the solution, followed by 3.1 g of N-(3-dimethylaminopropyl)-N′-ethylcarbodiimide hydrochloride (EDC HCl). The reaction mixture was stirred for 4 hours at laboratory temperature. The reaction mixture was diluted with 10 mL of dichloromethane (DCM) and extracted with 3×10 mL of distilled water. The organic phase was dried over Na₂SO₄ and concentrated under vacuum to 5 mL and crystallised in the freezer. The precipitated crystals were filtered off, washed with cold CHCl₃ and dried under vacuum. 2.6 g of Ma-β-Ala-cyclooctylamine monomer were obtained. Characterisation by HPLC on a Chromolith High Resolution RP18e column showed a single peak with a retention time of 11.3 min at 220 nm. Other monomers of type (T) were prepared by an analogous reaction described in this Example.

### Example 33

### Preparation of Raft-poly(HPMA-co-Ma-β-Ala-TT)-TT Statistical Copolymer with Terminal Reactive Group by RAFT Polymerisation

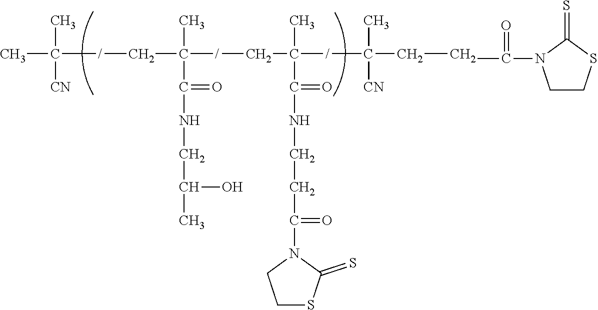

The polymer precursor Raft-poly(HPMA-co-Ma-β-Ala-TT)-TT containing TT reactive groups both along the chain and at the same time a TT group at the end of the polymer chain was prepared using RAFT (reversible addition-fragmentation chain-transfer)-copolymerisation. 1.5 g of HPMA (90% mol) was dissolved in 12.9 mL of tert-butanol, and 301 mg of Ma-β-Ala-TT (10% mol) dissolved in 2.8 mL of DMAc (dimethylacetamide), 6.53 mg of 2-[1-cyano-1-methyl-4-oxo-4-(2-thioxo-thiazolidin-3-yl)-butylazo]-2-methyl-5-oxo-5-(2-thioxothiazolidin-3-yl)-pentanenitrile and 2.76 mg of 2,2′-azobis(4-methoxy-2,4-dimethylvaleronitrile) were added to the solution and the solution was transferred to a polymerisation ampoule. The mixture was bubbled with argon for 10 min and then the ampoule was sealed. The polymerisation reaction was carried out at 40° C. (24 h). The polymer precursor Raft-poly(HPMA-co-Ma-β-Ala-TT)-TT was isolated by precipitation into acetone:diethyl ether (3:1), filtered off, washed with acetone and diethyl ether, and dried under vacuum. The terminal trithiocarbonate groups were removed according to a procedure previously published by Perrier, S., P. Takolpuckdee, and C. A. Mars, Reversible addition-fragmentation chain transfer polymerization: End group modification for functionalized polymers and chain transfer agent recovery. Macromolecules, 2005. 38(6): p. 2033-2036. Using this procedure, the polymer precursor poly(HPMA-co-Ma-β-Ala-TT)-TT was obtained with molecular weight Mw=52,000 g/mol, polydispersity D=1.15 and containing 9.6 mol % of reactive thiazolidine-2-thione (TT) groups, based on the total number of monomer units.

### Example 34

### Preparation of Raft-poly(HPMA-co-Ma-β-Ala-tetradecylamine)-tetradecylamine (Conjugate 19)

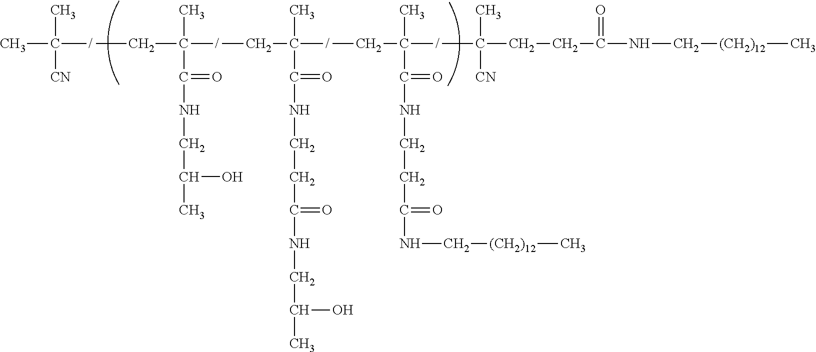

The polymer precursor Raft-poly(HPMA-co-Ma-β-Ala-TT)-TT (100 mg, Mw=52,000 g/mol, 9.6 mol % TT), prepared in Example 32 and tetradecylamine (3.3 mg) was dissolved in 0.50 mL of DMSO. N,N-diisopropylethylamine (DIPEA) (5.2 μL) was then added and the reaction mixture was stirred for 4 h at laboratory temperature. Subsequently, 1-amino-propan-2-ol (10 μl) was added to the solution and the reaction mixture was stirred for 10 min. Then, the polymer conjugate Raft-poly(HPMA-co-Ma-j-Ala-tetradecylamine)-tetradecylamine was isolated by precipitation into acetone:diethyl ether (3:1), filtered off, washed with acetone and diethyl ether and dried under vacuum. The total content of hydrophobic co-monomer unit (derived from N-[3-(tetradecylamino)-3-oxo-propyl]-2-methyl-prop-2-enamide) and tetradecylamine at the end of the polymer chain of 1.7 mol % was determined in the hydrolysate of the sample (6N HCl, 115° C., 16 h) using HPLC with a fluorescence detector (Ex. 229 nm, Em. 490 nm) on a Chromolith C18 column by the method of pre-column derivatisation with o-naphthalenedialdehyde. The content of copolymer units derived from N-2-hydroxypropylamide-2-methyl-prop-2-enamide was 7.9 mol %.

The conjugate molecular weight Mw 68,000 and dispersity 1.18 were determined by SEC equipped with a multi-angle scattering, viscometer and RI detector.

### Example 35

### Preparation of Poly(2-methyl-oxazoline-co-N′-tetradecyl-N-ethyl-butanediamide-co-N-ethyl-N′-quinuclidin-3-yl-butanediamide) Polymer Conjugate—Conjugate 20

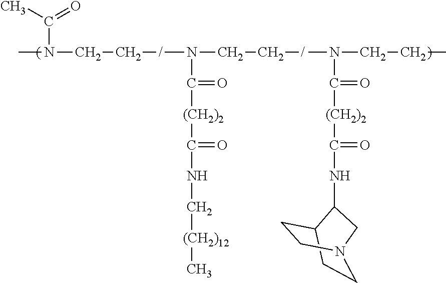

2-methyl-2-oxazoline (8.6 g, 101 mmol), 3-(4,5-dihydrooxazol-2-yl)-N-tetradecyl-propanamide (1.35 g, 4 mmol), 3-(4,5-dihydrooxazol-2-yl)-N-quinuclidin-3-yl-propanamide (1.0 g, 4 mmol) and 2-phenyl-2-oxazolinium tetrafluoroborate initiator (39.6 mg, 168 mol, [M]/[I]=600) were dissolved in a flask under an argon atmosphere in 36.3 mL of freshly distilled sulfolane such that the monomer concentration was 3 M. The reaction mixture was stirred at 60° C. in a glove box for 2 weeks, and then stopped overnight with solid sodium azide (33 mg, 504 mol). The polymerisation mixture was diluted with distilled water (15 mL) and dialysed against the same solvent (MWCO 3 kDa). Subsequently, the polymer was isolated by lyophilisation. The resulting polymer was obtained as a yellowish powder (yield 74% by weight).

The molecular weight of the conjugate Mw 14,200 and dispersity 1.11 were determined using a Shimadzu HPLC equipped with multi-angle scattering, viscometry and differential refractor detectors in PBS buffer. The content of co-monomer units was determined in the hydrolysate of the sample (6N HCl, 115° C., 16 h) using HPLC with a fluorescence detector (Ex. 229 nm, Em. 490 nm) on a Chromolith C18 column by the method of pre-column derivatisation with o-naphthalenedialdehyde. The sample contained 2.4 mol % co-monomer units derived from tetradecylamine and 2.3 mol % co-monomer units derived from quinuclidine.

### Example 36

### Preparation of Poly(2-methyl-oxazolin)-tetradecylamide Polymer Conjugate—Conjugate 21

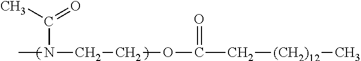

Conjugate 20 was prepared under argon by mixing 3 mmol of tetradecyl bromide initiator with 50 mmol of methyloxazoline monomer in 15 mL of chloroform at 0° C. for 1 h. The mixture was then allowed to react at 70° C. for two days. Polymerisation was stopped by adding at least a ten-fold excess of tetradecylamine relative to the amount of initiator and then maintaining the mixture at 70° C. for 24 h. After triple precipitation in diethyl ether, dialysis in water (benzoylated cellulose membrane from Aldrich with a molecular weight limit (MWCO) of 1,200 g/mol and drying in vacuum, the polymer was obtained in 79% yield.

### Example 37

### Preparation of Poly(2-methyl-oxazoline-co-N′-tetradecyl-N-ethyl-butandiamid-co-N-ethyl-N′-quinuclidin-3-yl-butandiamid-co-N-ethyl-N′-[2-[5-[(4R)-2-oxo-1,3,3a,4,6,6a-hexahydrothieno[3,4-d]imidazol-4-yl]pentanoylamino]ethyl]butandiamid)) Polymer Conjugate—Conjugate 20

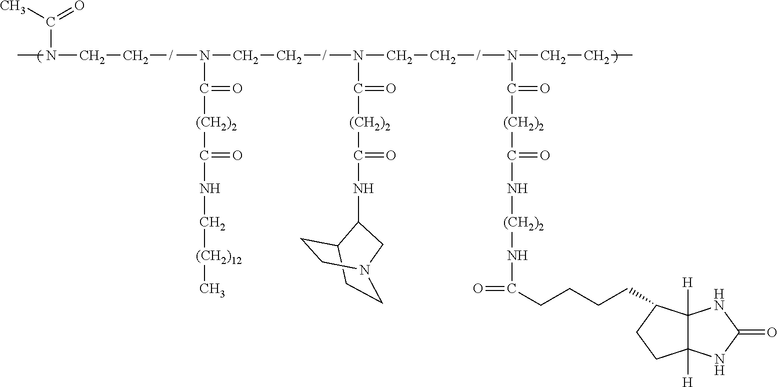

The polymer precursor poly(2-methyl-oxazoline-co-N-ethyl-4-oxo-4-(2-thioxothiazolidin-3-yl)butanamide) (1 g, Mw=12,400 g/mol, Ð=1.13, 3.8% mol TT), prepared according to Example 21, was dissolved in 5.0 mL of DMSO and 147 uL (0.03 mg) of a stock solution of tetradecylamine in CHCl₃ (1.02 mg/500 uL CHCl₃), 62.5 uL (0.15 mg) quinuclidine-3-amine (1.2 mg/500 uL CHCl₃) and 196.4 uL (0.55 mg) N-(2-aminoethyl)-5-[(4R)-2-oxo-1,3,3a,4,6,6a-hexahydrothieno[3,4-d]imidazol-4-yl]pentanamide (1.4 mg/500 uL CHCl₃) was added to the solution. N,N-diisopropylethylamine (DIPEA) (15 μL) was then added and the reaction mixture was stirred for 4 h at laboratory temperature. The crude product was isolated by precipitation into acetone:diethyl ether (3:1), filtered off, washed with acetone and diethyl ether and dried under vacuum. The content of co-monomer units (containing tetradecylamine 1.2 mol %, quinuclidin-3-amine 0.8 mol %) was determined in the hydrolysate of the sample (6N HCl, 115° C., 16 h) using HPLC with a fluorescence detector (Ex. 229 nm, Em. 490 nm) on a Chromolith C18 column by the method of pre-column derivatisation with o-naphthalenedialdehyde. The content of biotin units was 1.8 mol %. The molecular weight of conjugate Mw 16,200 and dispersity 1.15 were determined using a Shimadzu HPLC equipped with multi-angle scattering, viscometer and differential refractor detectors in PBS buffer.

1-20. (canceled)

21. A polymer conjugate for blocking of non-specific interactions in biological media, which is a statistical linear copolymer of the general formula (XXX) 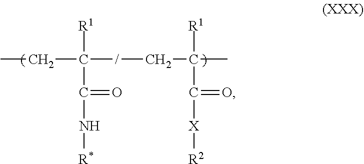
wherein
R¹ is independently selected from H and CH₃;
R* is —CH₂—CH(OH)—CH₃ or H;
R² is selected from the group consisting of —NH—(CH₂)_{b}—CH₃; —NH—(CH₂)_{b}—(CH═CH—CH₂)ₐ—(CH₂)_{b}—CH₃; —S—(CH₂)_{b}—(CH═CH—CH₂)ₐ—(CH₂)_{b}—CH₃; —O—C(═O)—(CH₂)_{b}—CH₃; —NH—C(═O)—(CH₂)_{b}—CH₃; phenyl, —S—(CH₂)ₐ—CH—((CH₂)_{b}—CH₃)₂; —O—(CH₂)_{b}—CH₃; —O—(CH₂)ₐ—C—((CH₂)_{b}—CH₃)₂; —NH—(CH₂)ₐ—C—((CH₂)_{b}—CH₃)₂; —O—(CH₂)_{b}—(CH═CH—CH₂)ₐ—(CH₂)_{b}—CH₃; and —O—C(═O)—(CH₂)ₐ—CH—((CH₂)_{b}—CH₃)₂; wherein a is an integer from 0 to 4, b is an integer from 4 to 17;
X is a covalent bond or a linker of the formula —NH—(CH₂)ₙ—C(═O)—; —NH—(CH₂—CH₂—O)ₒ—CH₂—CH₂—C(═O)—; —O—(CH₂—CH₂—O)ₒ—CH₂—CH₂—C(═O)—; or —NH—(CH₂)_{q}(C(═O)—NH—(CH₂)ᵣ)ₚ C(═O)—; wherein n is an integer from 1 to 7; o is an integer from 1 to 15; p, q and r are independently selected from the group consisting of 1, 2, and 3;
wherein if X is not a covalent bond, then the group R² or R is attached via its end —S— or —NH— or —O— group or via a carbon atom of the phenyl group to the end carbonyl group of the X linker;
wherein the —CH₂— groups of X may be further independently substituted with one or more of the same or different side chains of a natural amino acid;
wherein the end groups are selected from the group consisting of:
—C(CH₃)₂—CN;
—C(CH₃)(CH₂CH₃)—CN; 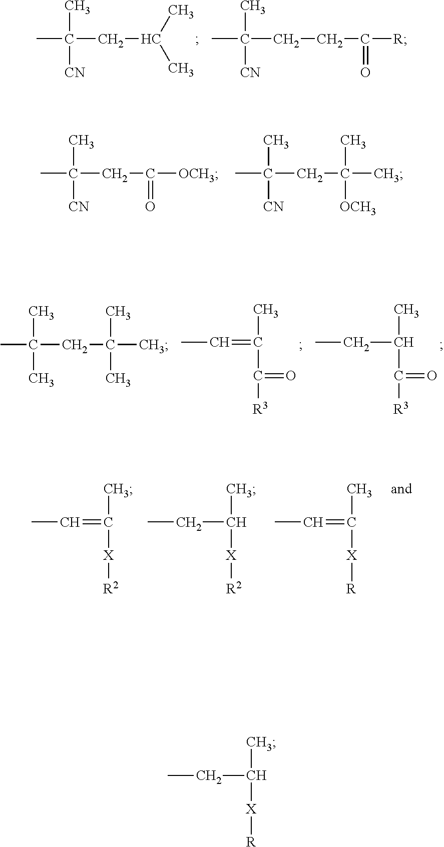
wherein R³ is selected from the group consisting of —NH—CH₂—CH(OH)—CH₃; —NH—CH₂CH₂—OH; —NH—CH₂CH₂CH₂—OH; —NH—C(CH₂OH)₃; —NH—CH(CH₂OH)₂; —NH—CH₂CH₂—N⁺(CH₃)₃Cl⁻; —O—CH₂CH₂—OH; —O—CH—(CH₂CH₂O)₂—H; —O—C—(CH₂CH₂O)₃—H; —O—CH₂CH₂—N⁺(CH₃)₃Cl⁻;
wherein R is selected from the group consisting of —OH, 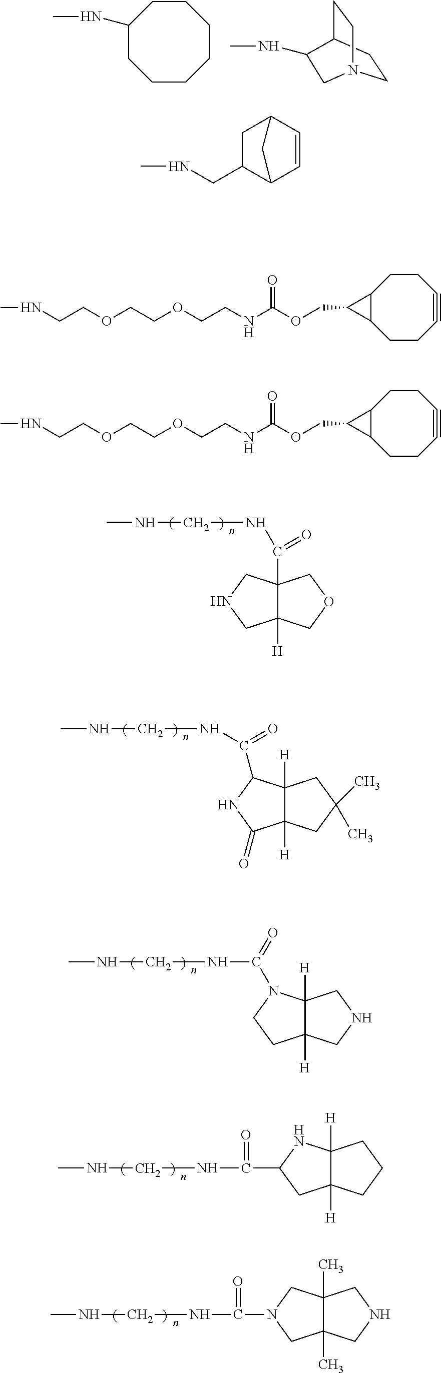 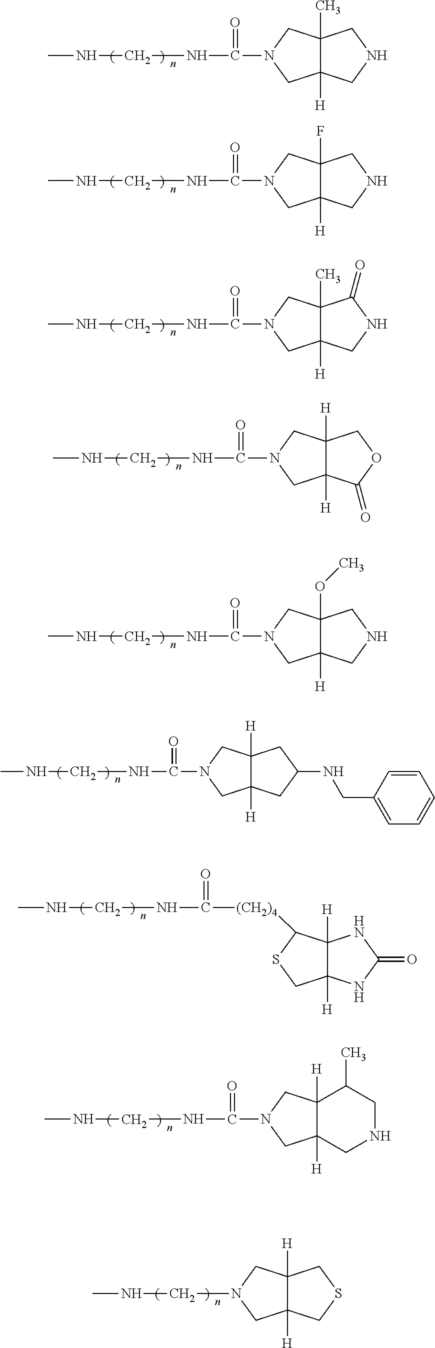 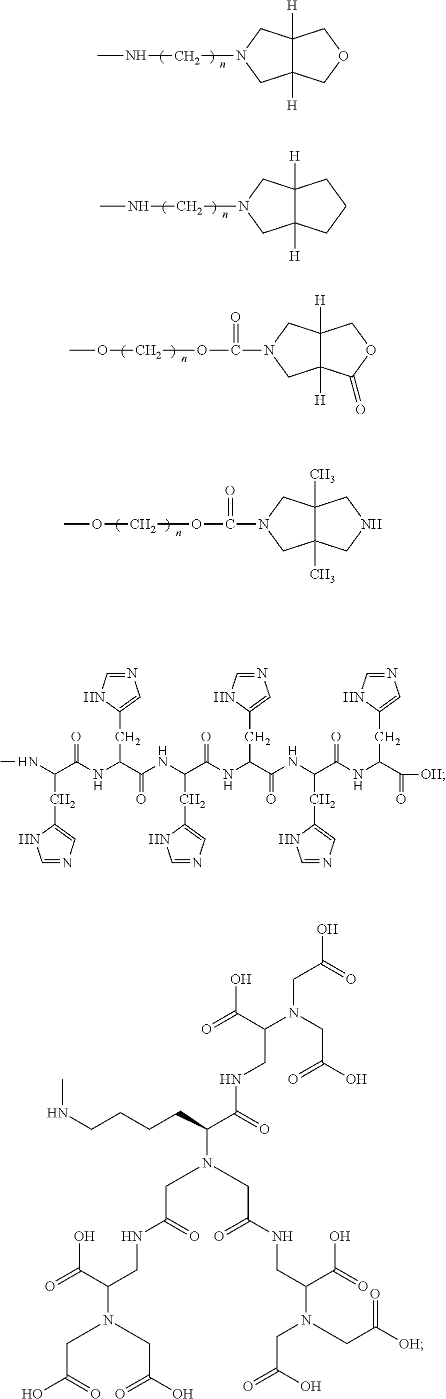 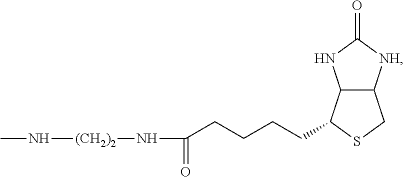
wherein n is an integer from 1 to 5;
wherein the content of the monomer units of the general formula (I) 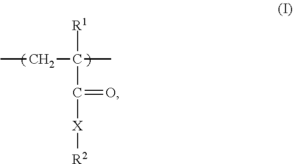
is in the range of from 0.5 to 10 mol %, based on the number of monomer units of the statistical linear copolymer;
and wherein the molecular weight of the polymer conjugate is in the range of from 5,000 to 500,000 g/mol, determined using HPLC equipped with multi-angle scattering, viscometer and differential refractive index detectors in PBS buffer.

22. The polymer conjugate according to claim 21, wherein X is —NH—(CH₂)ₙ—C(═O)—; wherein n is selected from the group consisting of 1, 2 and 3; and R² is selected from the group consisting of —NH—(CH₂)_{b}—CH₃; —NH—(CH₂)_{b}—(CH═CH—CH₂)ₐ—(CH₂)_{b}—CH₃; —O—(CH₂)_{b}—CH₃; wherein a is an integer from 0 to 4, b is an integer from 4 to 17;
wherein the R² group is attached via its end —NH— or —O— group to the end carbonyl group of the linker X.

23. The polymer conjugate according to claim 21, which further comprises from at least one monomer unit to 15 mol % of monomer units of the general formula (III), based on the number of monomer units of the statistical linear copolymer, 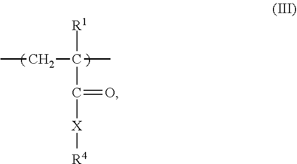
wherein X and R¹ are as defined in claim 21;
with the proviso that if X is not a covalent bond, the X group is attached via its end —NH— group or —O— group to the end carbonyl group of the monomer unit of the general formula (III);
R⁴ is selected from the group consisting of —NH—(CH₂)ₓ—CH₂(OH); —NH—(CH₂)_{y}—CH(OH)—CH₃; —NH—(CH₂)_{y}—CH(OH)—(CH₂)_{z}—CH₃; and —NH—C(═O)—CH₃; wherein x is an integer from 0 to 4, y is an integer from 0 to 3 and z is an integer from 1 to 4; with the proviso that if X is not a covalent bond, the R⁴ group is attached via its end —NH— group to the end carbonyl group of the linker X.

24. The polymer conjugate according to claim 23, comprising at least one monomer unit to 10 mol % of monomer units of the general formula (V), based on the number of monomer units of the statistical linear copolymer, 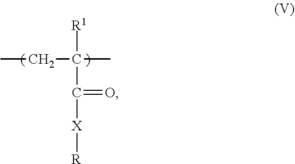
wherein
X, R, R¹ is as defined in claim 21;
with the proviso that if X is not a covalent bond, then X is attached via its end —NH— or —O— group to the terminal carbonyl group of the monomeric unit of the general formula (V).

25. The polymer conjugate according to claim 24, comprising a statistical linear copolymer of the general formula (B) 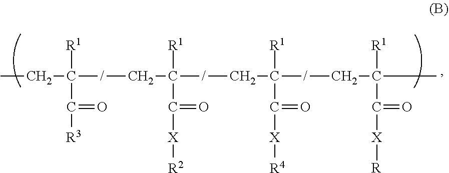
wherein
X, R, R¹, R², R³ are as defined in claim 21;
R⁴ is as defined in claim 23;
with the proviso that if X is not a covalent bond, the groups R², R⁴ and R are bound via their end —S— or —NH— or —O— group to the terminal carbonyl group of the linker X;
and wherein the end groups of the statistical linear copolymer of the general formula (B) are as defined in claim 21.

26. The polymer conjugate according to claim 21 wherein R¹ is CH₃ and R* is —CH₂—CH(OH)—CH₃.

27. A method of preparing the polymer conjugate according to claim 21, comprising the following steps:
i) polymerisation of the monomer (W) and of monomers selected from the group comprising monomers of general formulae (Y), (Z) and (T) 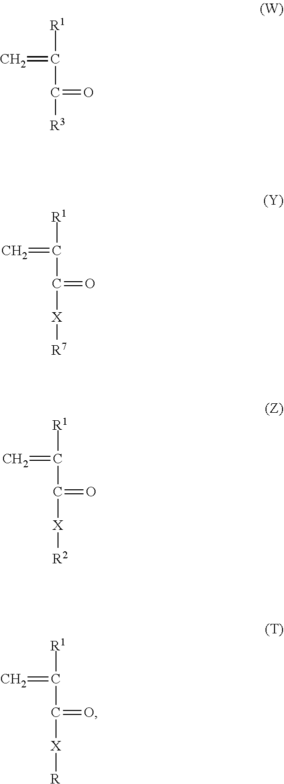
wherein X, R, R¹, R², R³ are as defined in claim 21;
R⁷ is selected from 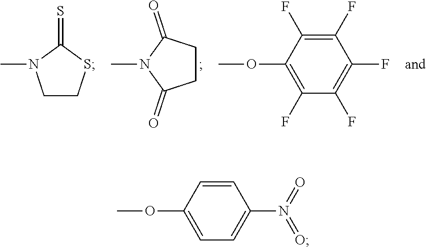
wherein step i) is performed by solution or precipitation radical copolymerisation or RAFT polymerisation; wherein one of the monomers is the monomer of the general formula (Y) and/or (Z) and/or (T), and wherein the total content of monomers of the general formulae (Y) and (Z) is in the range of from 0.5 to 20 mol %, based on the total number of monomers;
and wherein the reaction is carried out at a temperature in the range of from 30 to 100° C., and in a solvent preferably selected from the group comprising dimethyl sulfoxide, N,N-dimethylacetamide, N,N-dimethylformamide, sulfolane, methanol, ethanol, dioxane, tetrahydrofuran, propanol, isopropanol, tert-butanol, N-vinylpyrrolidone, acetone, water, and aqueous buffers or mixtures thereof,
to form the linear copolymer;
ii) optionally, conjugation of the R⁷ and/or thiazolidine-2-thione groups of the linear copolymer from step i) to a hydrophobic precursor of the general formula R²—H, wherein R² is as defined in claim 21, to form a polymer conjugate containing a hydrophobically active anchor attached via an amide or ester bond to the side chain of the monomer unit;
iii) optionally, conjugation of the R⁷ and/or thiazolidine-2-thione groups of the linear copolymer from step i) to a compound of the general formula R—H, wherein R is as defined in claim 21, to form a polymer conjugate comprising the group R attached via an amide or ester bond to the side chain of the monomer unit;
iv) optionally, removal of any unreacted reactive R⁷ groups and/or thiazolidine-2-thione groups by reaction with aminoalcohol, selected from the group comprising NH₂—(CH₂)ₓ—CH₂(OH); NH₂—(CH₂)_{y}—CH(OH)—CH₃; NH₂—(CH₂)_{y}—CH(OH)—(CH₂)_{z}—CH₃; wherein x is an integer from 0 to 4, y is an integer from 0 to 3 and z is from 1 to 4; preferably the aminoalcohol is 1-aminopropan-2-ol,
to form the polymer conjugate of the general formula (XXX).

28. An immunochemical method, comprising applying the polymer conjugate of claim 21 as a blocker of non-specific interactions of an analyte and other components of the analytical system with the solid phase.

29. The immunochemical method of claim 28, wherein the polymer conjugate is applied to capture specific antibodies or antigens or other molecules on the surface of the solid phase.
